(19) United States
(12) Patent Application Publication
(10) Pub. No.: US 2026/0020893 A1
(43) Pub. Date: Jan. 22, 2026

(54) SURGICAL INSTRUMENT

(71) Applicant: LIVSMED INC., Seongnam-si (KR)

(72) Inventor: Ik Jae JANG, Seongnam-si (KR)

(21) Appl. No.: 18/940,199

(22) Filed: Nov. 7, 2024

(30) Foreign Application Priority Data
Jul. 22, 2024 (KR) 10-2024-0096262

(51) Int. Cl. A61B 18/08; A61B 18/00; A61B 18/10

(52) U.S. Cl. CPC A61B 18/085; A61B 18/10; A61B 2018/00077; A61B 2018/00172; A61B 2018/00202; A61B 2018/00595; A61B 2018/00601; A61B 2018/00642; A61B 2018/00672; A61B 2018/00702; A61B 2018/00767; A61B 2018/00791; A61B 2018/00827; A61B 2018/00922

(57) ABSTRACT

Provided is a surgical instrument including an end tool rotatable in at least one direction and including a first jaw and a second jaw opposite to the first jaw, a manipulation part configured to control a rotational motion of the end tool, and a connection part connecting the end tool to the manipulation part, wherein at least one of the first jaw and the second jaw includes an electrode formed thereon and configured to receive power for a predetermined time period to be Joule heated and perform cauterization on a tissue, and the manipulation part includes a processor configured to obtain a momentary current value corresponding to the received power at each of at least two or more time points among a plurality of time points within the predetermined time period and control the power so that the cauterization is performed on the basis of a parameter calculated based on the momentary current values.

### CROSS-REFERENCE TO RELATED APPLICATION

This application is based on and claims priority under 35 USC § 119 to Korean Patent Application No. 10-2024-0096262, filed on Jul. 22, 2024, in the Korean Intellectual Property Office, the disclosure of which is incorporated by reference herein in its entirety.

### BACKGROUND

### 1. Field

The present disclosure relates to a surgical instrument, and more particularly, to a surgical instrument that indirectly controls electrode temperature by utilizing changes in current according to the amount of moisture in the tissue.

### 2. Description of the Related Art

Temperature control is crucial when using an electrode to remove only moisture from the tissue. When the temperature is set appropriately, it is possible to effectively remove only the moisture while preserving the physical or chemical characteristics of the tissue, but, when the temperature is not set correctly, it may result in incomplete moisture removal or alterations to the tissue's physical or chemical characteristics.

Meanwhile, to remove moisture from the tissue using an electrode, a closed-loop control system can be used, in which the temperature is monitored and adjusted in real-time through a temperature sensor and the control system to ensure the tissue's temperature does not deviate from a set range, but the need for a separate temperature sensor somewhat decreases usability.

The aforementioned background technology is technical information possessed by the inventor for derivation of the present disclosure or acquired by the inventor during the derivation of the present disclosure, and is not necessarily prior art disclosed to the public before the application of the present disclosure.

### SUMMARY

The present disclosure is directed to providing a surgical instrument. The problem to be solved by the present disclosure is not limited to the problems mentioned above, and other problems and advantages of the present disclosure, which are not mentioned, will be understood by the following description, and will be more clearly understood by the embodiments of the present disclosure. In addition, it will be appreciated that the problems and advantages to be solved by the present disclosure may be realized by means and combinations thereof indicated in the claims.

Additional aspects will be set forth in part in the description which follows and, in part, will be apparent from the description, or may be learned by practice of the presented embodiments of the disclosure.

A first aspect of the present disclosure may provide a surgical instrument including an end tool rotatable in at least one direction and including a first jaw and a second jaw opposite to the first jaw, a manipulation part configured to control a rotational motion of the end tool, and a connection part connecting the end tool to the manipulation part, wherein at least one of the first jaw and the second jaw includes an electrode formed thereon and configured to receive power for a predetermined time period to be Joule heated and perform cauterization on a tissue, and the manipulation part includes a processor configured to obtain a momentary current value corresponding to the received power at each of at least two or more time points among a plurality of time points within the predetermined time period and control the power so that the cauterization is performed on the basis of a parameter calculated based on the momentary current values.

In an embodiment, the processor may be further configured to control the power so that the electrode is heated in an open-loop manner.

In an embodiment, the processor may be further configured to control at least one of a voltage level and a pulse-width modulation (PWM) duty of the power.

In an embodiment, the processor may be further configured to determine a characteristic of the tissue in contact with the electrode based on the parameter and control the power based on the characteristic.

As an example, the characteristic may include a moisture state having at least one state that occurs during a process of removing moisture contained in the tissue. Specifically, the at least one state may include at least one of a first state in which a temperature of the moisture contained in the tissue increases, a second state in which the moisture evaporates, and a third state in which the temperature of the moisture increases. At this time, a first rate of change of the temperature of the moisture in the first state may be greater than a second rate of change of the temperature of the moisture in the third state.

For example, the processor may be further configured to determine the moisture state to be the first state based on a result of comparing the parameter with a first threshold value. As another example, the processor may be further configured to determine the moisture state to be the second state based on a result of comparing the parameter with a first threshold value and a second threshold value less than the first threshold value, As another example, the processor may be further configured to determine the moisture state to be the third state based on a result of comparing the parameter with a second threshold value.

In an embodiment, the processor may be further configured to determine whether control of the power is necessary based on the moisture state and control the power based on a result of the determination.

In an embodiment, the processor may be further configured to repeatedly calculate, determine, and control until the moisture state is determined to be a preset target state, and terminate the supply of the power in response to the moisture state being determined to be the preset target state.

In an embodiment, the processor may be further configured to supply initial power to the electrode, obtain an initial current value corresponding to the initial power, select a standard profile based on the initial current value, and determine the power to be supplied to the electrode based on the standard profile.

In an embodiment, the processor may be further configured to predict a thickness of the tissue based on the initial current value and select the standard profile corresponding to the thickness of the tissue.

In an embodiment, the processor may be further configured to calculate an amount of change in the parameter over time, and increase or decrease the power based on the amount of change being different from the standard profile by a predetermined value or more.

In an embodiment, the processor may be further configured to predict a temperature of the tissue based on the parameter.

In an embodiment, the manipulation part may include a current sensor disposed on an electric wire electrically connecting the electrode to the power board, and configured to measure a current value.

In an embodiment, the electrode may be a ceramic heating element.

In an embodiment, the end tool may further include a blade configured to, upon completion of the cauterization, cut the tissue while moving from a proximal end to a distal end of the first jaw.

In an embodiment, the manipulation part may further include a battery configured to store electrical energy, and a power board configured to convert the stored electrical energy to supply the power to the electrode.

In an embodiment, the surgical instrument may be a hand-held type device.

A second aspect of the present disclosure provides a device including a memory in which at least one program is stored, and a processor configured to perform calculations by executing the at least one program, wherein the processor may obtain a momentary current value corresponding to power supplied to an electrode at each of at least two or more time points among a plurality of time points included within a predetermined time period, and may control the power so that cauterization is performed on the basis of a parameter calculated based on the momentary current values.

In addition, other methods and devices for implementing the present disclosure, and a computer-readable recording medium having recorded thereon a program for executing the method may be further provided.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram for describing an example of a system for driving a surgical instrument according to an embodiment;

FIG. 2 is a block diagram illustrating an example of a user terminal according to an embodiment.

FIGS. 3 to 6 are views illustrating examples of use of a surgical instrument according to an embodiment of the present disclosure.

FIG. 7 is a perspective view illustrating a surgical instrument according to an embodiment of the present disclosure.

FIG. 8 is a perspective view illustrating a surgical instrument according to another embodiment of the present disclosure.

FIGS. 9 to 14 are views illustrating an end tool of the surgical instrument of FIG. 7.

FIG. 15 is a perspective view illustrating an end tool hub of the surgical instrument of FIG. 7.

FIGS. 16 and 17 are cut-away perspective views of the end tool hub of FIG. 15.

FIGS. 18 and 19 are perspective views illustrating the end tool hub of FIG. 15.

FIG. 20 is a side view illustrating the end tool hub of FIG. 15 and a guide tube.

FIG. 21 is a plan view illustrating the end tool hub of FIG. 15 and the guide tube.

FIGS. 22A and 22B are a set of perspective and cut-away perspective views illustrating an actuation hub of the surgical instrument of FIG. 7.

FIG. 23 is a view illustrating a state in which the guide tube, a blade wire, and a blade are mounted on the actuation hub illustrated in the cut-away perspective view of FIG. 22.

FIG. 24 is an exploded perspective view illustrating the end tool of the surgical instrument of FIG. 7.

FIG. 25 is a perspective view illustrating a first jaw of the end tool of the surgical instrument of FIG. 7.

FIG. 26 is a perspective view illustrating a second jaw of the end tool of the surgical instrument of FIG. 7.

FIG. 27 is a perspective view illustrating a first jaw pulley of the surgical instrument of FIG. 7.

FIGS. 28A and 28B are plan views illustrating opening and closing motions of the first jaw of the end tool of the surgical instrument of FIG. 7.

FIGS. 29A and 29B are plan views illustrating opening and closing motions of the second jaw of the end tool of the surgical instrument of FIG. 7.

FIGS. 30A and 30B are plan views illustrating opening and closing motions of the first jaw and the second jaw of the end tool of the surgical instrument of FIG. 7.

FIGS. 31 and 32 are plan views illustrating opening and closing motions of the end tool of the surgical instrument of FIG. 7.

FIGS. 33 to 35 are partial cross-sectional views illustrating the operation of the blade of the end tool of the surgical instrument of FIG. 7.

FIG. 36 is a flowchart of a method of controlling power according to an embodiment of the present disclosure.

FIG. 37 is a block diagram of a surgical instrument according to an embodiment of the present disclosure.

FIG. 38 is a flowchart of a method of controlling power based on moisture states according to an embodiment of the present disclosure.

FIG. 39 is a flowchart of a method of determining power to be supplied to an electrode based on a standard profile according to an embodiment of the present disclosure.

FIG. 40 is a flowchart of a method of controlling power according to another embodiment of the present disclosure.

FIGS. 41A to 42B are perspective views illustrating the operation of a sealing button of the surgical instrument shown in FIG. 7.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, following embodiments will be described in detail with reference to the accompanying drawings, and when the following embodiments are described with reference to the drawings, the same or corresponding components are given the same reference numerals, and repetitive descriptions thereof will be omitted.

As the present embodiments allow for various modifications, particular embodiments will be illustrated in the drawings and further described in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

In the drawings, for convenience of description, sizes of components may be exaggerated or reduced. For example, since the size and thickness of each component shown in the drawings are arbitrarily indicated for convenience of description, the following embodiment is not necessarily limited to what is illustrated.

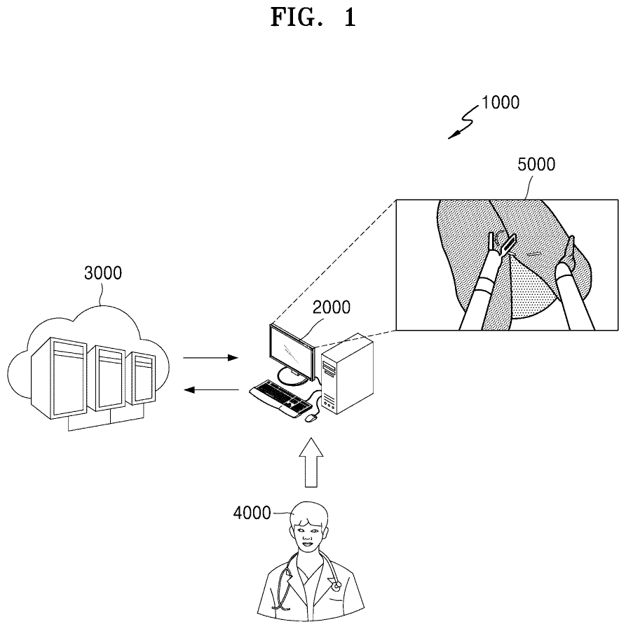

FIG. 1 is a diagram for describing an example of a system for driving a surgical instrument according to an embodiment.

Referring to FIG. 1, a system 1000 includes a user terminal 2000 and a server 3000. For example, the user terminal 2000 and the server 3000 may be connected by a wired or wireless communication method to send and receive data (e.g., a moisture state of the tissue corresponding to a current gradient, whether power control is necessary based on the moisture state of the tissue, a preset target state of the tissue, and a standard profile corresponding to an initial current value, and the like).

For convenience of description, in FIG. 1, the system 1000 is illustrated as including the user terminal 2000 and the server 3000, but the present disclosure is not limited thereto. For example, the system 1000 may include another external device (not shown), and operations of the user terminal 2000 and the server 3000 to be described later may be implemented by a single device (e.g., the user terminal 2000 or the server 3000) or a plurality of devices.

The user terminal 2000 may include a display device and a device for receiving user input (e.g., a keyboard, a mouse, or the like), and may be a computing device including a memory and a processor. For example, the display device may be implemented as a touch screen and may receive user input. For example, the user terminal 2000 may correspond to a notebook personal computer (PC), a desktop PC, a laptop computer, a tablet computer, a smartphone, and the like, but the present disclosure is not limited thereto.

The server 3000 may be a device that communicates with an external device (not shown) including the user terminal 2000. As an example, the server 3000 may be a device that stores various pieces of data, including momentary current values obtained over a predetermined time period, a parameter calculated based on the momentary current values, a moisture state of the tissue corresponding to the parameter, whether power control is necessary based on the moisture state of the tissue, a preset target state of the tissue, and a standard profile corresponding to an initial current value, and the like.

Alternatively, the server 3000 may be a computing device that includes a memory and a processor and has its own computing capabilities. As an example, the server 3000 may perform at least some of the operations of the user terminal 2000, which will be described later. For example, the server 3000 may be a cloud server, but the present disclosure is not limited thereto.

The user terminal 2000 may obtain momentary current values corresponding to power supplied to an electrode for a predetermined time period and control the power so that cauterization of the tissue is performed based on a parameter that is calculated on the basis of the momentary current values. For example, the user terminal 2000 may obtain a current value corresponding to the power supplied to the electrode, which will be described later. The current value corresponding to the power may refer to a measured value of a current flowing through a path through which the power is supplied to the electrode. For example, the current value corresponding to the power may refer to a current flowing through an electric wire that electrically connects the electrode to a manipulation part (or components included in the manipulation part) to be described later. The tissue to be cauterized includes a structure formed by multiple tissues. For example, the term “tissue” as used herein may be used in a sense that includes blood vessels.

Meanwhile, the user terminal 2000 may control the power so that cauterization of the tissue is performed based on the parameter calculated on the basis of the momentary current values. As an example, the user terminal 2000 may control the power in an open-loop manner so that the electrode is heated. Specifically, the manner in which the system is controlled can be divided into a closed-loop manner or an open-loop manner (in other words, a non-feedback loop method). In this case, the closed-loop manner utilizes an output signal of the system as feedback to directly affect an input of the system, while the open-loop manner controls the output of the system by generating a control signal using only a reference input without feeding back the output signal of the system to the input. In other words, the user terminal 2000 may control the power in a manner in which the result of heating the electrode (e.g., the temperature of the electrode or the tissue) does not affect the power control. As another example, the user terminal 2000 may control at least one of a voltage level and a pulse-width modulation (PWM) duty of the power.

Meanwhile, the user terminal 2000 may calculate a parameter using the obtained momentary current values, determine characteristics of the tissue in contact with the electrode based on the parameter, and control the power based on the characteristics of the tissue. As an example, the characteristics of the tissue may include a moisture state of the tissue. For example, the moisture state may include at least one state occurring during a process of removing moisture from the tissue. In this case, the moisture contained in the tissue may include blood. In addition, the moisture state may include a first state in which the temperature of the moisture contained in the tissue increases, a second state in which the moisture evaporates, and a third state in which the temperature of the moisture increases. That is, the user terminal 2000 may determine the moisture state of the tissue in contact with the electrode to be one of the first state in which the temperature of the moisture increases, the second state in which the moisture evaporates, and the third state in which the moisture is removed.

As an example, the user terminal 2000 may determine the moisture state to be the first state based on the result of comparing the parameter with a first threshold value. In addition, the user terminal 2000 may determine the moisture state to be the second state based on the result of comparing the parameter with the first threshold value and a second threshold value less than the first threshold value. In addition, the user terminal 2000 may determine the moisture state to be the third state based on the result of comparing the parameter with the second threshold value.

Meanwhile, the user terminal 2000 may determine whether power control is necessary based on the moisture state and may control the power based on the determination result. In addition, the user terminal 2000 may repeatedly calculate, determine, and control until the moisture state is determined to be the preset target state, and may terminate the supply of the power in response to the moisture state being determined to be the preset target state.

In an embodiment, the user terminal 2000 may supply initial power to the electrode, obtain an initial current value corresponding to the initial power, select a standard profile based on the initial current value, and determine power to be supplied to the electrode based on the standard profile. As an example, the standard profile may refer to a profile of parameters that are expected according to the initial current value and a predicted thickness of the tissue. The initial current value refers to a first value among the momentary current values obtained during the above-described predetermined time period. In addition, as described above, the initial current value corresponding to the initial power may refer to a current value in the path through which the initial power is supplied to the electrode. For example, the initial current value corresponding to the initial power may refer to a measured value of the current flowing through the electric wire that electrically connects the electrode to a manipulation part (or components included in the manipulation part) when the initial power is supplied to the electrode.

Meanwhile, the user terminal 2000 may predict the thickness of the tissue based on the initial current value and select a standard profile corresponding to the thickness of the tissue. Specifically, the user terminal 2000 may calculate a change in the parameter over time and increase or decrease the power based on the change in the parameter being different from the standard profile by a predetermined value or more. In addition, in an embodiment, the user terminal 2000 may predict the temperature of the tissue based on the parameter.

Meanwhile, the user terminal 2000 may control power applied to an electrode of an end tool or drive a surgical instrument through an application installed on the user terminal 2000. Here, the application may be a software program installed for activities of a user 4000 to drive the surgical instrument. For example, through the application, the user 4000 may perform various activities described above.

Meanwhile, the user terminal 2000 may output an image 5000 indicating a motion of the surgical instrument driven based on a motion of the user 4000. For example, the user terminal 2000 may drive the surgical instrument based on a moisture state of the tissue corresponding to a parameter, whether power control is necessary based on the moisture state of the tissue, a preset target state of the tissue, a standard profile corresponding to the initial current value, and the like. In addition, the user terminal 2000 may output the image 5000 indicating a motion of the surgical instrument driven based on the moisture state of the tissue. The user 4000 can intuitively understand the motion of the surgical instrument according to the motion of the user 4000 through the image 5000 representing the motion of the surgical instrument and operate the surgical instrument more accurately.

Meanwhile, for convenience of description, throughout the specification, the user terminal 2000 has been described as obtaining momentary current values corresponding to power supplied to the electrode during a predetermined time period, calculating parameter using the momentary current values, determining a moisture state of the tissue in contact with the electrode based on the parameter, controlling the power based on the moisture state so that cauterization is performed on the tissue, predicting a thickness of the tissue, and predicting a temperature of the tissue, but the present disclosure is not limited thereto For example, at least some of the operations performed by the user terminal 2000 may be performed by the server 3000.

In other words, at least some of the operations of the user terminal 2000 to be described later may be performed by the server 3000. For example, the server 3000 may obtain momentary current values corresponding to power supplied to the electrode, calculate a parameter using the momentary current values, determine a moisture state of the tissue in contact with the electrode based on the parameter, control the power based on the moisture state so that cauterization is performed on the tissue, predict a thickness of the tissue, and predict a temperature of the tissue.

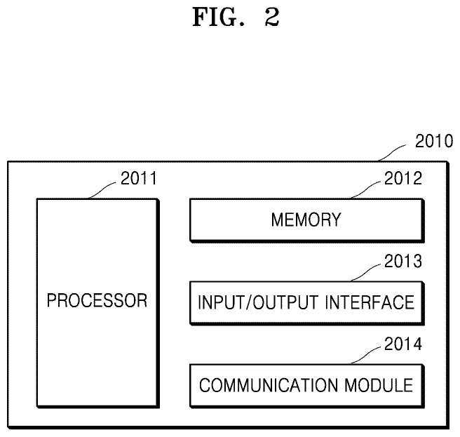

FIG. 2 is a block diagram illustrating an example of a user terminal according to an embodiment.

Referring to FIG. 2, a user terminal 2010 includes a processor 2011, a memory 2012, an input/output interface 2013, and a communication module 2014. For convenience of description, only components related to the present disclosure are illustrated in FIG. 2. Accordingly, other general-purpose components in addition to the components illustrated in FIG. 2 may be further included in the user terminal 2010. In addition, it will be apparent to those skilled in the art related to the present disclosure that the processor 2011, the memory 2012, the input/output interface 2013, and the communication module 2014 illustrated in FIG. 2 may be implemented as independent devices.

The processor 2011 may process instructions of a computer program by performing a basic arithmetic operation, a logic operation, and an input/output operation. Here, the instructions may be provided from the memory 2012 or an external device (e.g., the server 3000 or the like). In addition, the processor 2011 may control overall operations of the other components included in the user terminal 2010.

The processor 2011 may obtain momentary current values corresponding to power supplied to the electrode for a predetermined time period and control the power so that cauterization of the tissue is performed based on a parameter that is calculated on the basis of the momentary current values. For example, the processor 2011 may obtain a momentary current value corresponding to the power supplied to the electrode at each of at least two or more time points among a plurality of time points included within a predetermined time period.

The member for supplying power to the electrode of the surgical instrument may be formed as a handle-shaped manipulation member, but is not limited thereto, and may be modified and implemented in various shapes to achieve the same purpose. For example, a portion of the member may be provided in the form of a handle, and the other portions thereof may be provided in different forms, such as a clutch button. In addition, a finger insertion tube may be further formed so as to allow the surgical operator's finger to be inserted therethrough and fixed to facilitate manipulation of the surgical tool.

Meanwhile, the processor 2011 may control the power so that cauterization of the tissue is performed based on the above-described parameter. As an example, the processor 2011 may control the power so that the electrode is heated in an open-loop manner. As another example, the processor 2011 may control at least one of a voltage level and a PWM duty cycle of the power.

Meanwhile, the processor 2011 may calculate a parameter using momentary current values, determine a moisture state of the tissue in contact with the electrode based on the parameter, and control the power based on the moisture state. For example, the moisture state may include a plurality of states according to a process of removing the moisture contained in the tissue. At this time, the moisture contained in the tissue may include blood. In addition, the moisture state may include a first state in which the temperature of the moisture increases, a second state in which the moisture evaporates, and a third state in which the temperature of the moisture increases but at a slower rate than in the first state. That is, the processor 2011 may determine the moisture state of the tissue in contact with the electrode to be any one of the first state in which the temperature of the moisture increases, the second state in which the moisture evaporates, and the third state in which the temperature of the moisture increases.

As an example, the processor 2011 may determine the moisture state to be the first state based on the result of comparing the parameter with a first threshold value. In addition, the processor 2011 may determine the moisture state to be the second state based on the result of comparing the parameter with the first threshold value and a second threshold value that is less than the first threshold value. In addition, the processor 2011 may determine the moisture state to be the third state based on the result of comparing the parameter with the second threshold value.

Meanwhile, the processor 2011 may determine whether power control is necessary based on the moisture state, and control the power based on the determination result. In addition, the processor 2011 may repeatedly obtain, determine, and control until the moisture state is determined to be a preset target state, and may terminate the supply of the power in response to the moisture state being determined to be the preset target state.

In an embodiment, the processor 2011 may supply initial power to the electrode, obtain an initial current value corresponding to the initial power, select a standard profile based on the initial current value, and determine power to be supplied to the electrode based on the standard profile.

Meanwhile, the processor 2011 may predict a thickness of the tissue based on the initial current value and select a standard profile corresponding to the thickness of the tissue. Specifically, the processor 2011 may calculate an amount of change in the parameter over time, and increase or decrease the power based on the amount of change in the parameter being different from the standard profile by a predetermined value or more. In addition, in an embodiment, the processor 2011 may predict the temperature of the tissue based on the parameter.

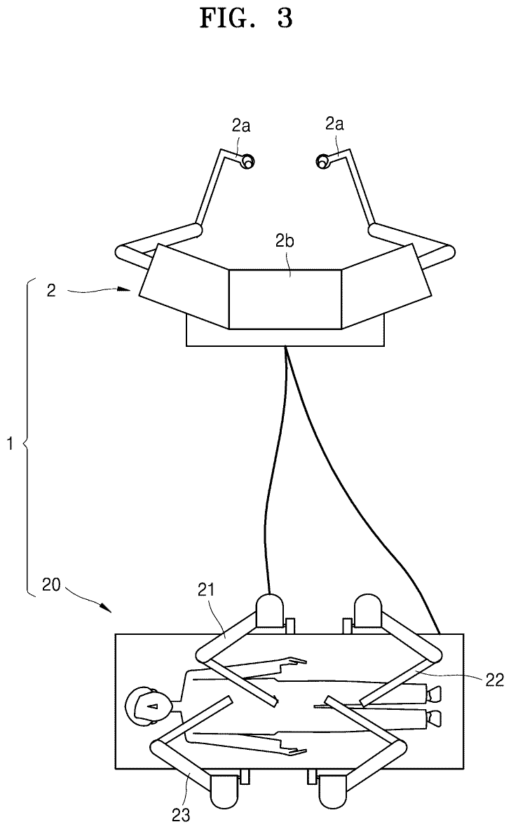

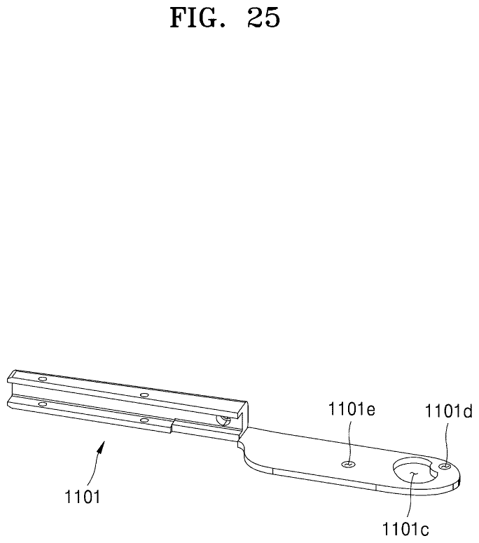

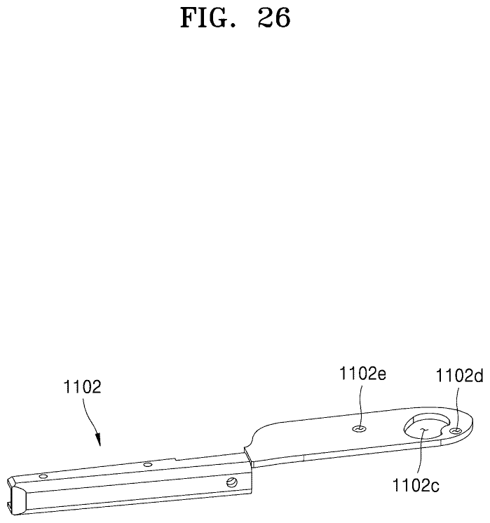

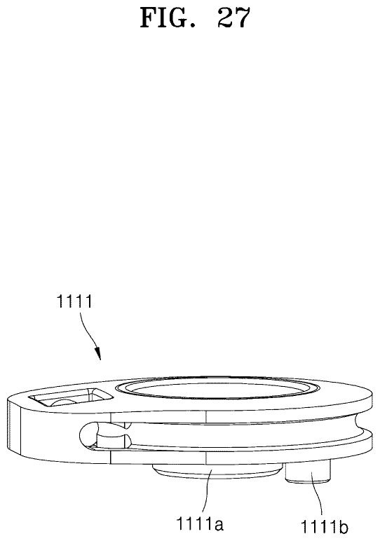

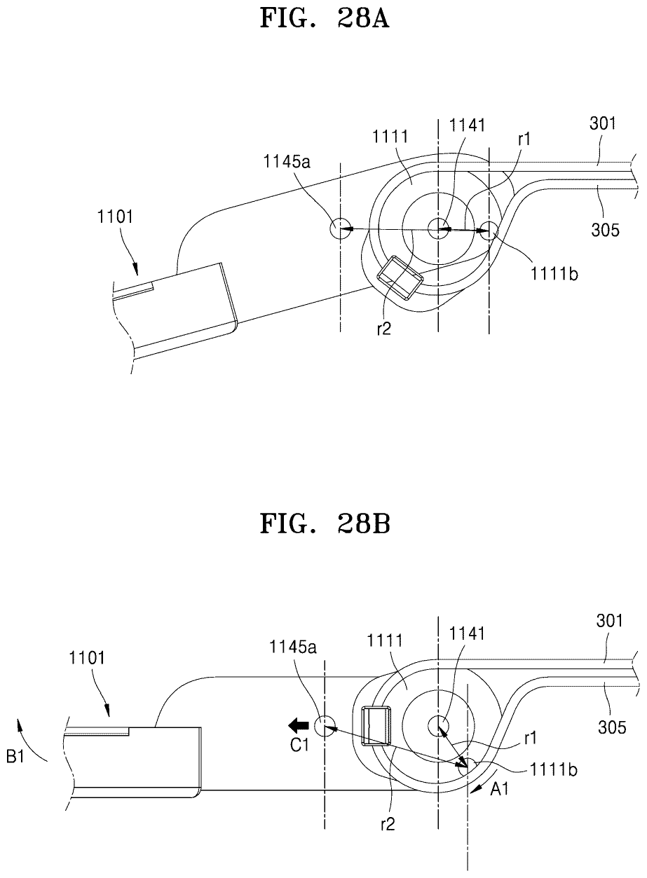

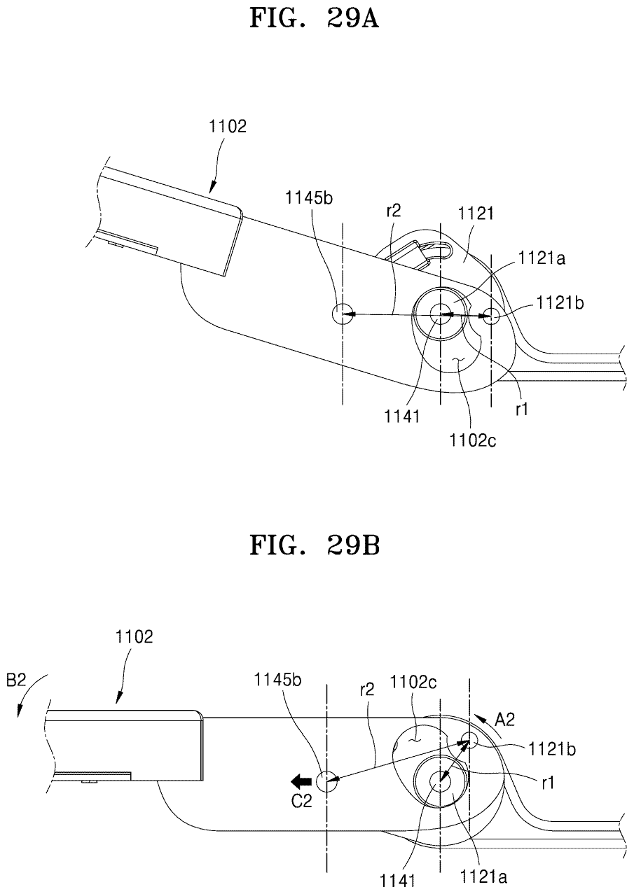

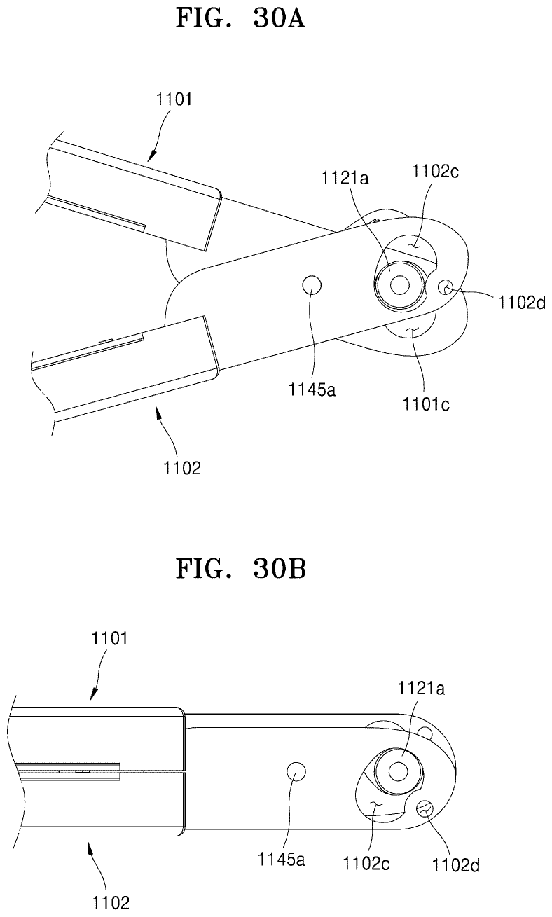

Specific examples in which the processor 2011 according to an embodiment operates will be described with reference to FIGS. 3 to 42.

The processor 2011 may be implemented in an array of multiple logic gates, or in a combination of a universal microprocessor and a memory that stores a program executable in the microprocessor. For example, the processor 2011 may include a general-purpose processor, a central processing unit (CPU), a microprocessor, a digital signal processor (DSP), a controller, a microcontroller, a state machine, or the like. In some environments, the processor 2011 may include an application-specific semiconductor (ASIC), a programmable logic device (PLD), a field programmable gate array (FPGA), or the like. For example, the processor 2011 may refer to a combination of processing devices such as, for example, a combination of a DSP and a microprocessor, a combination of a plurality of microprocessors, a combination of one or more microprocessors in conjunction with a DSP core, or a combination of any other such configuration.

The memory 2012 may include any non-transitory computer-readable recording medium. In an example, the memory 2012 may include a permanent mass storage device such as a random access memory (RAM), a read-only memory (ROM), a disk drive, a solid state drive (SSD), a flash memory, or the like. In another example, the permanent mass storage device such as a ROM, SSD, a flash memory, a disk drive, or the like may be a separate permanent storage device which is distinguishable from the memory. In addition, an operating system (OS) and at least one program code (e.g., a code for the processor 2011 to perform operations to be described later with reference to FIGS. 3 to 42) may be stored in the memory 2012.

These software components may be loaded from a computer-readable recording medium separate from the memory 2012. The separate computer-readable recording medium may be a recording medium that may be directly connected to the user terminal 2010, and may include, for example, a computer-readable recording medium, such as a floppy drive, a disk, a tape, a DVD/CD-ROM drive, a memory card, or the like. Alternatively, the software components may be loaded into the memory 2012 through the communication module 2014 instead of the computer-readable recording medium. For example, at least one program may be loaded into the memory 2012 based on a computer program (for example, a computer program for performing, by the processor 2011, operations to be described later with reference to FIGS. 3 to 42) installed by the files provided through the communication module 2014 by developers or a computer file distribution system that distributes the installation files of applications.

The input/output interface 2013 may be a member for an interface with a device (e.g., a keyboard, a mouse, or the like) for input or output, the member being connected to the user terminal 2010 or being included in the user terminal 2010. The input/output interface 2013 may be configured separately from the processor 2011, but the present disclosure is not limited thereto, and the input/output interface 2013 may be configured to be included in the processor 2011.

The communication module 2014 may provide a configuration or a function for the server 3000 and the user terminal 2010 to communicate with each other through a network. In addition, the communication module 2014 may provide a configuration or function for the user terminal 2010 to communicate with another external device. For example, a control signal, a command, data, or the like, which is provided according to the control of the processor 2011, may be transmitted to the server 3000 and/or an external device through the communication module 2014 and the network.

Meanwhile, although not shown in FIG. 2, the user terminal 2010 may further include a display device. For example, the display device may be implemented as a touch screen. Alternatively, the user terminal 2010 may be connected to an independent display device through a wired or wireless communication method to transmit/receive data to or from each other. For example, a video, an image, or the like of driving the surgical instrument may be provided through the display device by using driving information.

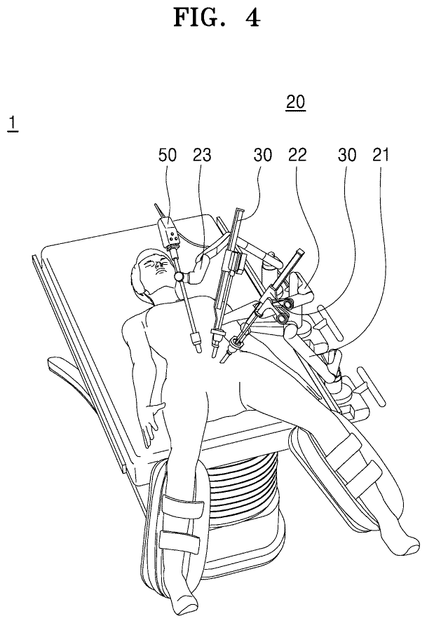

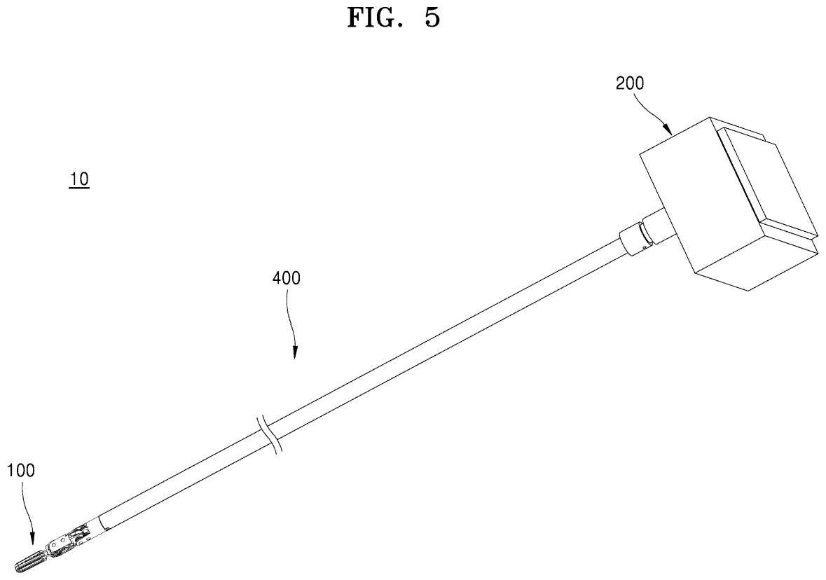

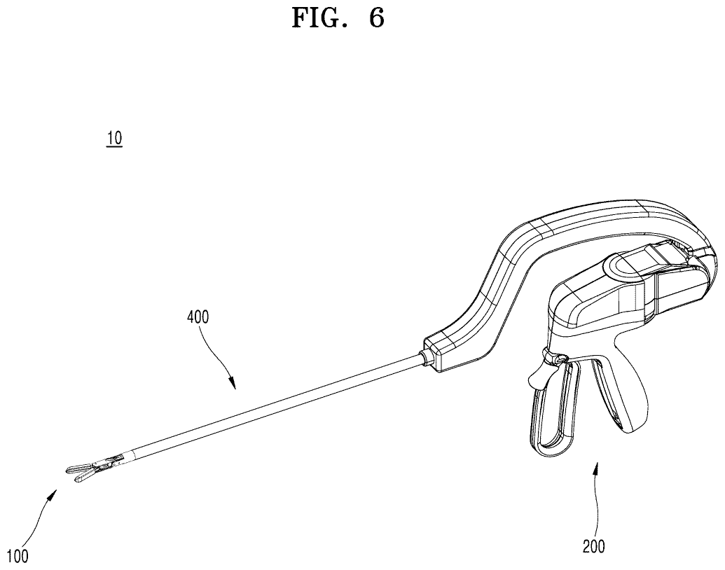

FIGS. 3 to 6 are views illustrating examples of use of an end tool 100 of a surgical instrument 10 according to an embodiment of the present disclosure. Here, FIG. 3 is a conceptual view illustrating a surgical robot system 1 on which the end tool 100 of the surgical instrument 10 according to an embodiment of the present disclosure is mounted. FIG. 4 is a perspective view illustrating a slave robot 20 of the surgical robot system 1 of FIG. 3, and FIG. 5 is a perspective view illustrating the surgical instrument 10 mounted on the slave robot 20 of FIG. 3. In addition, FIG. 6 is a perspective view illustrating an example of the surgical instrument 10, which is of a hand-held type and on which the end tool 100 according to an embodiment of the present disclosure is mounted.

Referring to FIGS. 3, 4, and 5, the surgical robot system 1 includes a master robot 2, the slave robot 20, and the surgical instrument 10.

The master robot 2 includes manipulating members 2a and a display member 2b, and the slave robot 20 includes one or more robot arm units 21, 22, and 23.

In detail, the master robot 2 includes the manipulating members 2a so that a surgical operator can grip and manipulate them respectively with both hands. In addition, an image captured through the laparoscope 50 is displayed as a screen image on the display member 2b of the master robot 2. In addition, a predetermined virtual manipulation plate may be displayed independently or displayed together with the image captured by the laparoscope 50 on the display member 2b. A detailed description of the arrangement, configuration, and the like of such a virtual manipulation plate will be omitted.

Meanwhile, the slave robot 20 may include one or more robot arm units 21, 22, and 23. Here, each of the robot arm units 21, 22, and 23 may be provided in the form of a module that can operate independently of each other, and in this case, an algorithm for preventing a collision between the robot arm units 21, 22, and 23 may be applied to the surgical robot system 1.

Here, two or more of the robot arm units 21, 22, and 23 may each have the surgical instrument 10 attached thereto, and one or more of the robot arm units 21, 22, and 23 may have the laparoscope 50 attached thereto. In addition, the surgical operator may select the robot arm unit 21, 22, or 23 to be controlled via the master robot 2. As described above, by directly controlling a total of three or more surgical instruments through the master robot 2, the surgical operator may accurately and freely control various tools according to the intention of the surgical operator without a surgical assistant.

Continuing to refer to FIG. 5, the surgical instrument 10 of the surgical robot system 1 may include the end tool 100, a manipulation part 200, and a connection part 400.

Here, the connection part 400 is formed in the shape of a hollow shaft, in which one or more wires may be accommodated, and may have one end portion to which the manipulation part 200 is coupled and another end portion to which the end tool 100 is coupled and serve to connect the manipulation part 200 to the end tool 100.

The manipulation part 200 is formed at one end portion of the connection part 400 and provides an interface capable of being coupled to the robot arm units 21, 22, and 23. Accordingly, when a user operates the master robot 2, a motor (not shown) of the robot arm units 21, 22, and 23 is operated so that the end tool 100 of the surgical instrument 10 can perform a motion corresponding thereto, and a driving force of the motor (not shown) is transmitted to the end tool 100 through the manipulation part 200. In other words, it may be described that the manipulation part 200 itself becomes an interface that connects between the surgical instrument 10 and the slave robot 20.

The end tool 100 is formed on another end portion of the connection part 400, and performs necessary motions for surgery by being inserted into a surgical site.

Meanwhile, the surgical instrument 10 may be a hand-held type device. Referring to FIG. 6, the hand-held type surgical instrument 10 may include the end tool 100, the manipulation part 200, and the connection part 400.

Here, the connection part 400 is formed in the shape of a hollow shaft, in which one or more wires (to be described later) may be accommodated, and may have one end portion to which the manipulation part 200 is coupled and another end portion to which the end tool 100 is coupled and serve to connect the manipulation part 200 to the end tool 100.

The manipulation part 200 is formed at the one end portion of the connection part 400 and provided as an interface to be directly controlled by a medical doctor, for example, a tongs shape, a stick shape, a lever shape, or the like, and when the medical doctor controls the manipulation part 200, the end tool 100, which is connected to the interface and inserted into the body of a surgical patient, performs a certain motion, thereby performing surgery. Here, the manipulation part 200 is illustrated in FIG. 6 as being formed in a handle shape that is rotatable while the finger is inserted therein, the concept of the present disclosure is not limited thereto, and various types of manipulation parts 200 that are connected to the end tool 100 and manipulate the end tool 100 may be possible.

The end tool 100 is formed on another end portion of the connection part 400, and performs necessary motions for surgery by being inserted into a surgical site.

The end tool 100 of the surgical instrument according to an embodiment of the present disclosure may be provided in the surgical instrument 10 of the surgical robot system 1 shown in FIGS. 3, 4, and 5, or may also be provided in the hand-held type surgical instrument 10 shown in FIG. 6.

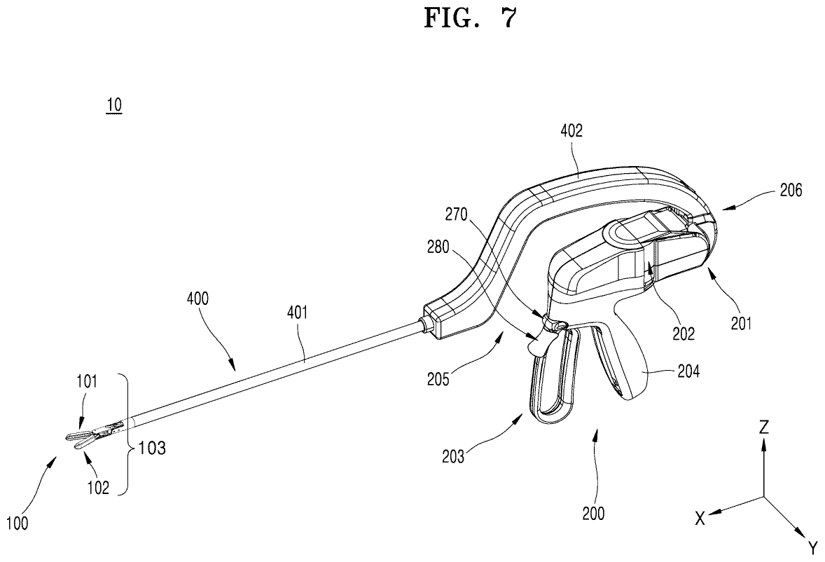

FIG. 7 is a perspective view illustrating a surgical instrument according to an embodiment of the present disclosure.

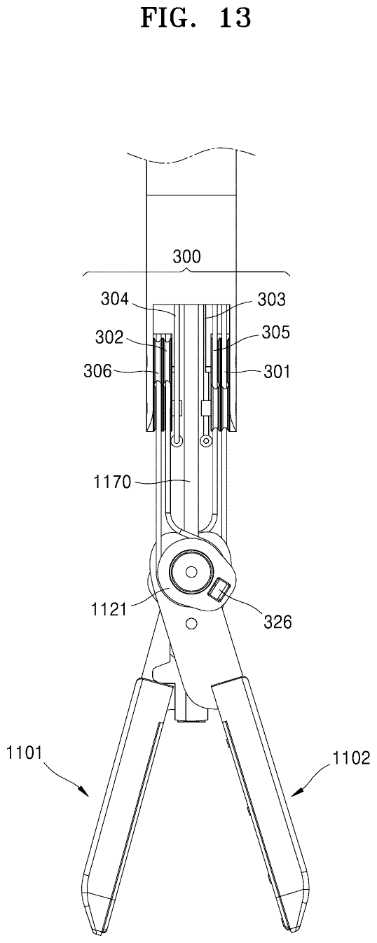

Referring to FIG. 7, a surgical instrument 10 according to an embodiment of the present disclosure includes an end tool 100, a manipulation part 200, a power transmission part (see 300 in FIG. 13), and a connection part 400.

Here, the connection part 400 is formed in the shape of a hollow shaft, and one or more wires and electric wires may be accommodated therein. The manipulation part 200 is coupled to one end portion of the connection part 400, the end tool 100 is coupled to another end portion thereof, and the connection part 400 may serve to connect the manipulation part 200 to the end tool 100. Here, the connection part 400 of the surgical instrument 10 according to an embodiment of the present disclosure includes a straight part 401 and a bent part 402, wherein the straight part 401 may be formed at a side coupled to the end tool 100, and the bent part 402 is formed at a side to which the manipulation part 200 is coupled. As such, since the end portion of the connection part 400 at the side of the manipulation part 200 is formed to be bent, a pitch manipulation part 201, a yaw manipulation part 202, and an actuation manipulation part 203 may be formed along an extension line of the end tool 100 or adjacent to the extension line. In other words, it may be said that the pitch manipulation part 201 and the yaw manipulation part 202 are at least partially accommodated in a concave portion formed by the bent part 402. Due to the above-described shape of the bent part 402, the shapes and motions of the manipulation part 200 and the end tool 100 may be further intuitively matched with each other.

Meanwhile, a plane on which the bent part 402 is formed may be substantially the same as a pitch plane, that is, an XZ plane of FIG. 7. As such, as the bent part 402 is formed on the plane substantially the same as the XZ plane, interference with the manipulation part 200 may be reduced. Of course, for intuitive motions of the end tool 100 and the manipulation part 200, any form other than the XZ plane may be possible.

Meanwhile, the surgical instrument 10 may be a hand-held type device. Referring to FIG. 7, the hand-held type surgical instrument 10 may be driven using electrical energy stored in a battery without a separate connector or external power source. That is, as an example, the surgical instrument 10 may be implemented as a hand-held type device by including a battery. An embodiment in which the surgical instrument 10 is driven using electrical energy stored in a battery will be described later.

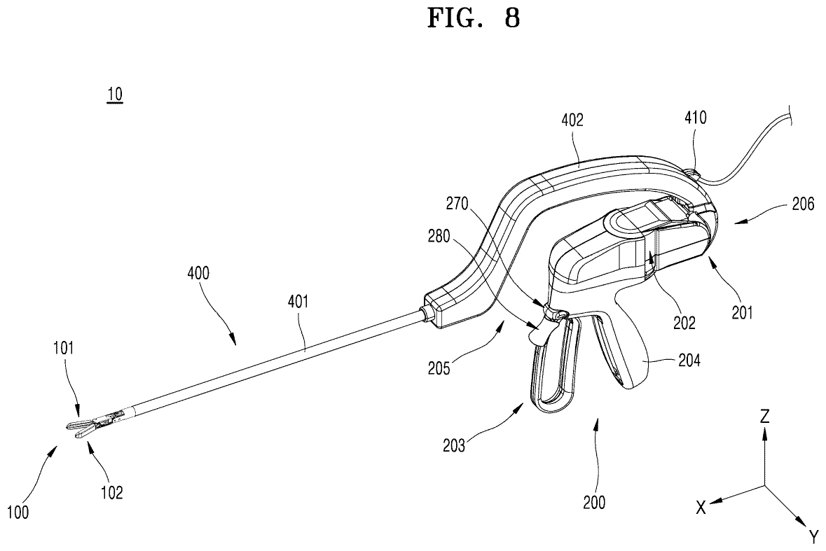

FIG. 8 is a perspective view illustrating a surgical instrument according to another embodiment of the present disclosure.

Referring to FIG. 8, a connector 410 may be further formed in the bent part 402. The connector 410 may be connected to an external power source (not shown), and the connector 410 may be connected to a jaw 103 to transmit electrical energy supplied from the external power source (not shown) to the jaw 103. Here, the connector 410 may be of a bipolar-type having two electrodes, or the connector 410 may be of a monopolar type having one electrode.

Referring to FIGS. 7 and 8 again, the manipulation part 200 is formed at the one end portion of the connection part 400 and provided as an interface to be directly controlled by a medical doctor, such as, a tongs shape, a stick shape, a lever shape, or the like, and when the medical doctor controls the manipulation part 200, the end tool 100, which is connected to the interface and inserted into the body of a surgical patient, performs a certain motion, thereby performing surgery. Here, the manipulation part 200 is illustrated in FIG. 7 as being formed in a handle shape that is rotatable while the finger is inserted therein, the concept of the present disclosure is not limited thereto, and various types of manipulation parts that are connected to the end tool 100 and manipulate the end tool 100 may be possible.

The end tool 100 is formed on another end portion of the connection part 400, and performs necessary motions for surgery by being inserted into a surgical site. In an example of the above-described end tool 100, as shown in FIG. 7, a pair of jaws 101 and 102 for performing a grip motion may be used. However, the concept of the present disclosure is not limited thereto, and various devices for performing surgery may be used as the end tool 100. For example, a configuration of a cantilever cautery may also be used as the end tool 100. The end tool 100 is connected to the manipulation part 200 by the power transmission part 300, and receives a driving force of the manipulation part 200 through the power transmission part 300 to perform a motion necessary for surgery, such as gripping, cutting, suturing, or the like.

Here, the end tool 100 of the surgical instrument 10 according to an embodiment of the present disclosure is formed to be rotatable in at least one or more directions. For example, the end tool 100 may be configured to perform a pitch motion around a Y-axis of FIG. 7 while simultaneously performing a yaw motion and an actuation motion around a Z-axis of FIG. 7.

Here, each of the pitch, yaw, and actuation motions used in the present disclosure are defined as follows.

First, the pitch motion means a motion of the end tool 100 rotating in a vertical direction with respect to an extension direction of the connection part 400 (an X-axis direction of FIG. 7), that is, a motion rotating around the Y-axis of FIG. 7. In other words, the pitch motion means a motion of the end tool 100, which is formed to extend from the connection part 400, rotating vertically around the Y-axis with respect to the connection part 400.

Next, the yaw motion means a motion of the end tool 100 rotating in left and right directions, that is, a motion rotating around the Z-axis of FIG. 7, with respect to the extension direction of the connection part 400 (the X-axis direction of FIG. 7). In other words, the yaw motion means a motion of the end tool 100, which is formed to extend from the connection part 400, rotating horizontally around the Z-axis with respect to the connection part 400. That is, the yaw motion means rotating motions of two jaws 101 and 102, which are formed on the end tool 100, around the Z-axis in the same direction.

Meanwhile, the actuation motion means a motion of the end tool 100 rotating around the same shaft of rotation as that of the yaw motion, while the two jaws 101 and 102 rotating in the opposite directions so as to be closed or opened. That is, the actuation motion means rotating motions of the two jaws 101 and 102, which are formed on the end tool 100, in the opposite directions around the Z-axis.

The power transmission part 300 may serve to connect the manipulation part 200 to the end tool 100 to transmit the driving force of the manipulation part 200 to the end tool 100, and may include a plurality of wires, pulleys, links, sections, gears, and the like.

Meanwhile, for convenience of description, the plurality of wires, pulleys, and the like have been categorized as being included in the power transmission part 300, but the wires, pulleys, and the like on the end tool 100 side may be categorized as being included in the end tool 100, and those on the manipulation part 200 side may be categorized as being included in the manipulation part 200.

Hereinafter, intuitive driving of the surgical instrument 10 of the present disclosure will be described.

First, while holding a first handle 204 with the palm of the hand, the user may rotate the first handle 204 around the Y-axis (i.e., a rotation shaft 246) to perform a pitch motion, and rotate the first handle 204 around the Z-axis (i.e., a rotation shaft 243) to perform a yaw motion. In addition, the user may perform an actuation motion by manipulating the actuation manipulation part 203 in a state in which the thumb and the index finger are inserted into a hand ring-shaped actuation extension part formed at one end portion of the actuation manipulation part 203.

Here, in the surgical instrument 10 according to an embodiment of the present disclosure, when the manipulation part 200 is rotated in one direction with respect to the connection part 400, the end tool 100 is rotated in a direction that is intuitively the same as a manipulation direction of the manipulation part 200. In other words, when the first handle 204 of the manipulation part 200 is rotated in one direction, the end tool 100 is also rotated in a direction intuitively the same as the one direction, so that a pitch motion or a yaw motion is performed. Here, the phrase “intuitively the same direction” may be further explained as meaning that a direction of movement of the user's finger gripping the manipulation part 200 and a direction of movement of a distal end of the end tool 100 form substantially the same direction. Of course, “the same direction” as used herein may not be a perfectly matching direction on a three-dimensional coordinate, and may be understood to be equivalent to the extent that, for example, when the user's finger moves to the left, the distal end of the end tool 100 is moved to the left, and when the user's finger moves down, the end portion of the end tool 100 is moved down.

In addition, to this end, in the surgical instrument 10 according to an embodiment of the present disclosure, the manipulation part 200 and the end tool 100 are formed in the same direction with respect to a plane perpendicular to the extension axis (X-axis) of the connection part 400. That is, when viewed based on a YZ plane of FIG. 7, the manipulation part 200 is formed to extend in a positive (+) X-axis direction, and the end tool 100 is also formed to extend in the positive (+) X-axis direction. In other words, it may be said that a formation direction of the end tool 100 on one end portion of the connection part 400 is the same as a formation direction of the manipulation part 200 on another end portion of the connection part 400 on the basis of the YZ plane. Further, in other words, it may be said that the manipulation part 200 may be formed in a direction away from the body of a user holding the manipulation part 200, that is, in a direction in which the end tool 100 is formed. In other words, the first handle 204, the actuation manipulation part 203, or the like, which the user grips and moves to perform the actuation motion, the yaw motion, the pitch motion, and the like, is formed such that the portion that moves to perform each motion extends in the positive (+) X-axis direction beyond the center of rotation of each joint for that motion. In this manner, the manipulation part 200 may be configured in the same manner as the end tool 100 in which each moving portion is formed to extend in the positive (+) X-axis direction from the rotation center of a corresponding joint for the motion, and the manipulation direction of the user may be identical to an operation direction of the end tool 100 from the viewpoint of the rotation directions and the left and right directions. As a result, intuitively the same manipulation may be achieved.

In detail, in the case of the conventional surgical instrument, a direction in which a user manipulates the manipulation part is different from a direction in which the end tool is actually operated, that is, intuitively different from the direction in which the end tool is actually operated, and thus, a surgical operator may not easily intuitively manipulate the surgical instrument and may spend a long time to learn a skill of operating the end tool in desired directions, and in some cases, malfunctions may occur, which may cause damage to patients.

In order to address such problems, the surgical instrument 10 according to an embodiment of the present disclosure is configured such that the manipulation direction of the manipulation part 200 and the operation direction of the end tool 100 are intuitively identical to each other. To this end, the manipulation part 200 is configured similar to the end tool 100, that is, in the manipulation part 200, parts that are actually moved for actuation, yaw, and pitch motions extend respectively from rotation centers of corresponding joints in the positive (+) X-axis direction.

Hereinafter, the power transmission part 300 of the surgical instrument 10 of FIG. 7 will be described in more detail.

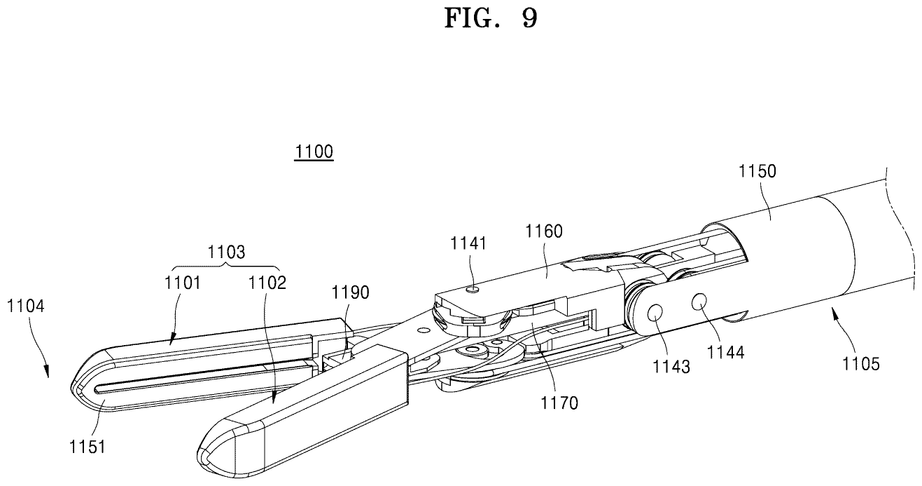

Referring to FIGS. 9 to 14 and the like, the power transmission part 300 of the surgical instrument 10 according to an embodiment of the present disclosure may include a wire 301, a wire 302, a wire 303, a wire 304, a wire 305, a wire 306, and a blade wire 307.

Here, the wire 301 and the wire 305 may be paired to serve as first jaw wires. The wire 302 and the wire 306 may be paired to serve as second jaw wires. Here, the components encompassing the wire 301 and the wire 305, which are first jaw wires, and the wire 302 and the wire 306, which are second jaw wires, may be referred to as jaw wires. In addition, the wire 303 and the wire 304 may be paired to serve as pitch wires.

In addition, the power transmission part 300 of the surgical instrument 10 according to an embodiment of the present disclosure may include a fastening member 321, a fastening member 322, a fastening member 323, a fastening member 324, a fastening member 326, and a fastening member 327 that are coupled to respective end portions of the wires to respectively couple the wires and the pulleys. Here, each of the coupling members may have various shapes as necessary, such as a ball shape, a tube shape, and the like.

Here, at an end tool 1100 side, the fastening member 321/fastening member 322 may serve as pitch wire-end tool fastening members, the fastening member 323 may serve as a first jaw wire-end tool fastening member, and the fastening member 326 may serve as a second jaw wire-end tool fastening member.

Further, at the manipulation part 200 side, the fastening member 324 may serve as a first jaw wire-manipulation part fastening member, and the fastening member 327 may serve as a second jaw wire-manipulation part fastening member. In addition, although not shown in the drawings, a pitch wire-manipulation part fastening member and a blade wire-manipulation part fastening member may be further formed at the manipulation part 200 side.

The coupling relationship between the wires, the coupling members, and the respective pulleys will be described in detail as follows.

First, the wires 301 and 305, which are first jaw wires, may be a single wire. The coupling member 323, which is a first jaw wire-end tool coupling member, is inserted at an intermediate point of the first jaw wire, which is a single wire, and the coupling member 323 is crimped and fixed, and then, both strands of the first jaw wire centered on the coupling member 323 may be referred to as the wire 301 and the wire 305, respectively.

Alternatively, the wires 301 and 305, which are first jaw wires, may also be formed as separate wires and connected by the coupling member 323.

In addition, by coupling the fastening member 323 to a pulley 1111, the wires 301 and 305 may be fixedly coupled to the pulley 1111. This allows the pulley 1111 to rotate as the wires 301 and 305 are pulled and released.

Meanwhile, the first jaw wire-manipulation part coupling member 324 may be coupled to another end portions of the wires 301 and 305, which are opposite to one end portions to which the coupling member 323 is coupled.

In addition, by coupling the first jaw wire-manipulation part fastening member 324 to a pulley 211, the wires 301 and 305 may be fixedly coupled to the pulley 211. As a result, when the pulley 211 is rotated by a motor or human power, the wire 301 and the wire 305 are pulled and released, allowing the pulley 1111 of the end tool 1100 to rotate.

In the same manner, the wires 302 and 306, which are second jaw wires, are respectively coupled to the coupling member 326, which is a second jaw wire-end tool coupling member, and the second jaw wire-manipulation part coupling member 327. In addition, the fastening member 326 is coupled to a pulley 1121, and the second jaw wire-manipulation part fastening member is coupled to a pulley 220. As a result, when the pulley 220 is rotated by a motor or a human force, the pulley 1121 of the end tool 1100 may be rotated as the wire 302 and the wire 306 are pulled and released.

In the same manner, the wire 304, which is a pitch wire, is coupled to the fastening member 321, which is a pitch wire-end tool fastening member, and the pitch wire-manipulation part fastening member (not shown). In addition, the wire 303, which is a pitch wire, is coupled to a fastening member 322, which is a pitch wire-end tool fastening member, and the pitch wire-manipulation part fastening member (not shown).

In addition, the fastening member 321 is coupled to a first pitch pulley part 1163a of an end tool hub 1160, the fastening member 322 is coupled to a second pitch pulley part 1163b of the end tool hub 1160, and the pitch wire-manipulation part fastening member (not shown) is coupled to a pulley 231. As a result, when the pulley 231 is rotated by a motor or human force, the wire 303 and the wire 304 are pulled and released, allowing the end tool hub 1160 of the end tool 1100 to rotate.

In describing the present disclosure, the part closer to the user side, i.e., the part closer to the manipulation part 200, is described as a proximal end, and the part farther from the user side, that is, the part closer to the end tool 1100, will be described as a distal end.

For example, referring to FIG. 9, the part of the end tool 1100 closer to the manipulation part 200 will be defined as a proximal end 1105 of the end tool 1100, and the part farther from the manipulation part 200, that is, the part closer to an end portion of the end tool 1100 will be defined and described as a distal end 1104 of the end tool 1100. In other words, the proximal end 1105 of the end tool 1100 may be described as the part closer to the connection part 400, and the distal end 1104 of the end tool 1100 may be described as the part farther from the connection part 400.

Meanwhile, one end portion of the blade wire 307 is coupled to a blade 1175 to be described later, and another end portion thereof is coupled to a cutting manipulation part 280 of the manipulation part 200. By the manipulation of the cutting manipulation part 280, a cutting motion may be performed as the blade wire 307 is moved from a proximal end 1105 toward a distal end 1104 of the end tool 1100, or the blade wire 307 may return from the distal end 1104 toward the proximal end 1105 of the end tool 1100.

At this time, at least a portion of the blade wire 307 may be accommodated in a guide tube 1170 to be described later. Accordingly, when the guide tube 1170 is bent in response to a pitch motion or yaw motion of the end tool 1100, the blade wire 307 accommodated therein may also be bent together with the guide tube 1170. The guide tube 1170 will be described in more detail later.

Hereinafter, the end tool 1100 of the surgical instrument 10 of FIG. 7 will be described in more detail.

FIGS. 9 to 14 are views illustrating the end tool of the surgical instrument of FIG. 7.

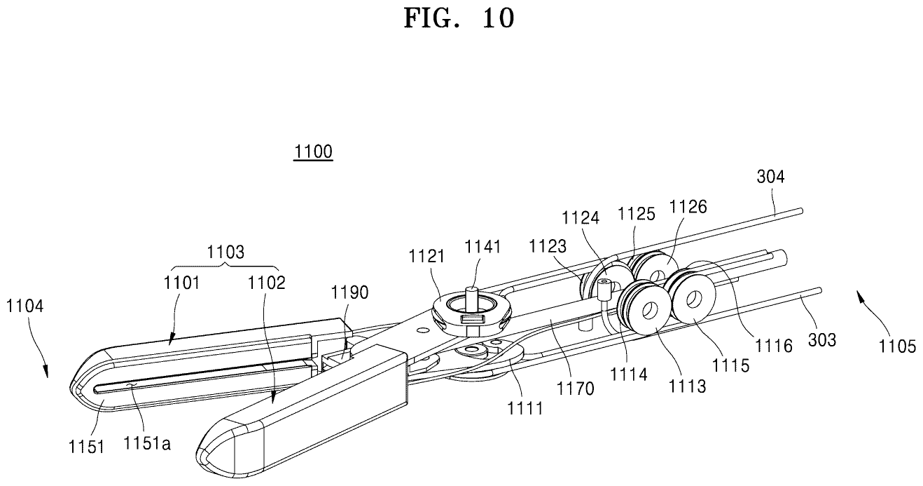

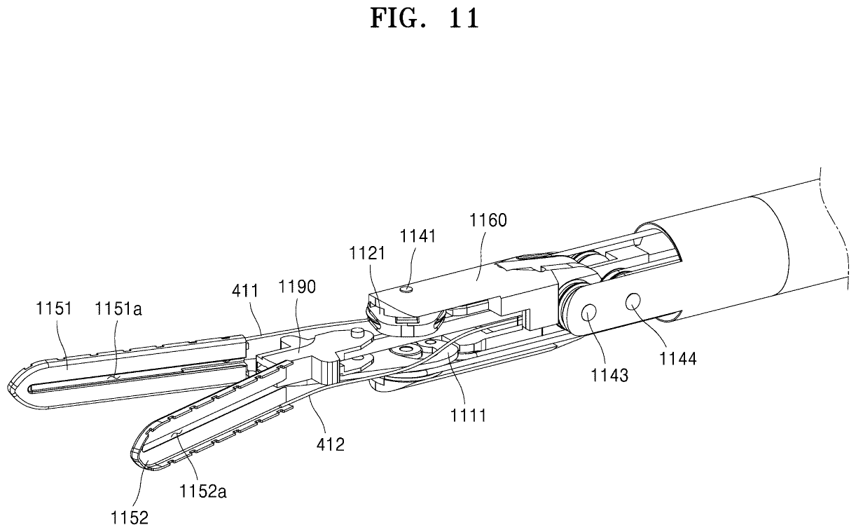

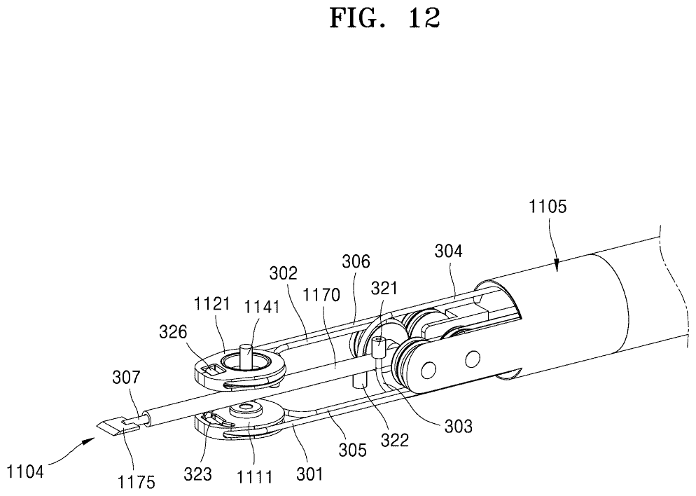

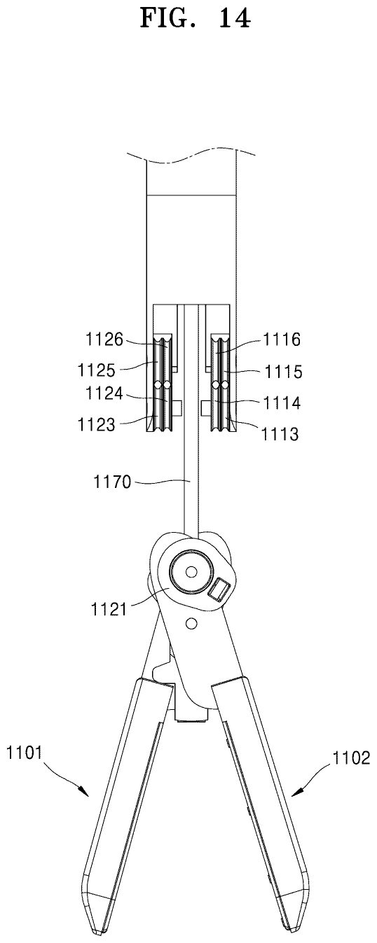

Here, FIG. 9 illustrates a state in which the end tool hub 1160 and a pitch hub 1150 are coupled, and FIG. 10 illustrates a state in which the end tool hub 1160 and pitch hub 1150 are removed. FIG. 11 illustrates a state in which a first jaw 1101 and a second jaw 1102 are removed, and FIG. 12 illustrates a state in which the first jaw 1101, the second jaw 1102, the pulley 1111, the pulley 1121, and the like are removed. Meanwhile, FIG. 13 is a view mainly illustrating the wires, and FIG. 14 is a view mainly illustrating the pulleys.

Referring to FIGS. 7 to 30 and the like, the end tool 1100 according to an embodiment of the present disclosure includes a pair of jaws for performing a grip motion, that is, the first jaw 1101 and the second jaw 1102. Here, each of the first jaw 1101 and the second jaw 1102, or a component encompassing the first jaw 1101 and the second jaw 1102 may be referred to as a jaw 1103.

Further, the end tool 1100 may include the pulley 1111, a pulley 1113, a pulley 1114, a pulley 1115, and a pulley 1116 associated with a rotational motion of the first jaw 1101. In addition, the end tool 1100 may include the pulley 1121, a pulley 1123, a pulley 1124, a pulley 1125, and a pulley 1126, which are associated with a rotational motion of the second jaw 1102.

Here, the pulleys facing each other are illustrated in the drawings as being formed parallel to each other, but the concept of the present disclosure is not limited thereto, and each of the pulleys may be variously formed with a position and a size suitable for the configuration of the end tool.

Further, the end tool 1100 according to an embodiment of the present disclosure may include the end tool hub 1160 and the pitch hub 1150.

A first rotation shaft 1141 to be described later may be inserted through the end tool hub 1160, and the pulley 1111 and the pulley 1121 axially coupled to the first rotation shaft 1141 and at least some of the first jaw 1101 and the second jaw 1102 coupled to the pulley 1111 and the pulley 1121 may be accommodated inside the end tool hub 1160. Here, in an embodiment of the present disclosure, a wire guide part 1168 serving as an auxiliary pulley is formed in the end tool hub 1160. That is, a first wire guide part 1168a and a second wire guide part 1168b for guiding paths of the wire 305 and the wire 302 may be formed in the end tool hub 1160. The wire guide parts 1168 of the end tool hub 1160 may serve as auxiliary pulleys and change the paths of the wires, and the first wire guide part 1168a and the second wire guide part 1168b of the end tool hub 1160 serving as auxiliary pulleys will be described in more detail later.

Meanwhile, the first pitch pulley part 1163a and the second pitch pulley part 1163b, which serve as end tool pitch pulleys, may be formed at one end portion of the end tool hub 1160. The wire 303 and the wire 304, which are pitch wires, are coupled to the first pitch pulley part 1163a and the second pitch pulley part 1163b, which serve as end tool pitch pulleys, and a pitch motion is performed while the end tool hub 1160 rotates around a third rotation shaft 1143.

The third rotation shaft 1143 and a fourth rotation shaft 1144 may be inserted through the pitch hub 1150, and the pitch hub 1150 may be axially coupled to the end tool hub 1160 by the third rotation shaft 1143. Accordingly, the end tool hub 1160 may be formed to be pitch-rotatable around the third rotation shaft 1143 with respect to the pitch hub 1150.

Further, the pitch hub 1150 may internally accommodate at least some of the pulley 1113, the pulley 1114, the pulley 1123, and the pulley 1124 that are axially coupled to the third rotation shaft 1143. Further, the pitch hub 1150 may internally accommodate at least some of the pulley 1115, the pulley 1116, the pulley 1125, and the pulley 1126 that are axially coupled to the fourth rotation shaft 1144.

One end portion of the pitch hub 1150 is connected to the end tool hub 1160, and another end portion of the pitch hub 1150 is connected to the connection part 400.

Here, the end tool 1100 according to an embodiment of the present disclosure may include the first rotation shaft 1141, the third rotation shaft 1143, and the fourth rotation shaft 1144. As described above, the first rotation shaft 1141 may be inserted through the end tool hub 1160, and the third rotation shaft 1143 and the fourth rotation shaft 1144 may be inserted through the pitch hub 1150.

The first rotation shaft 1141, the third rotation shaft 1143, and the fourth rotation shaft 1144 may be arranged sequentially from the distal end 1104 toward the proximal end 1105 of the end tool 1100. Accordingly, starting from the distal end 1104, the first rotation shaft 1141 may be referred to as a first pin, the third rotation shaft 1143 may be referred to as a third pin, and the fourth rotation shaft 1144 may be referred to as a fourth pin.

Here, the first rotation shaft 1141 may function as an end tool jaw pulley rotation shaft, the third rotation shaft 1143 may function as an end tool pitch rotation shaft, and the fourth rotation shaft 1144 may function as an end tool pitch auxiliary rotation shaft of the end tool 1100.

Here, each of the rotation shafts may include two shafts of a first sub-shaft and a second sub-shaft. Alternatively, it may be said that each of the rotation shafts is formed by being divided into two parts.

For example, the first rotation shaft 1141 may include two shafts of a first sub-shaft 1141a and a second sub-shaft 1141b. In addition, the third rotation shaft 1143 may include two shafts of a first sub-shaft 1143a and a second sub-shaft 1143b. In addition, the fourth rotation shaft 1144 may include two shafts of a first sub-shaft and a second sub-shaft.

Each of the rotation shafts is formed by being divided into two parts as described above to allow the guide tube 1170 to be described later to pass through the end tool hub 1160 and the pitch hub 1150. That is, the guide tube 1170 may pass between the first sub-shaft and the second sub-shaft of each of the rotation shafts. This will be described in more detail later. Here, the first sub-shaft and the second sub-shaft may be disposed on the same axis or may be disposed to be offset to a certain degree.

Meanwhile, it is illustrated in the drawings that each of the rotation shafts is formed by being divided into two parts, but the concept of the present disclosure is not limited thereto. That is, each of the rotation shafts is formed to be curved in the middle such that an escape path for the guide tube 1170 is formed.

Each of the rotation shafts 1141, 1143, and 1144 may be fitted into one or more pulleys, which will be described in detail below.

Meanwhile, the end tool 1100 may further include an actuation rotation shaft 1145. In detail, the first jaw 1101 and the second jaw 1102 may be axially coupled by the actuation rotation shaft 1145, and in this state, an actuation motion may be performed while the first jaw 1101 and the second jaw 1102 rotate around the actuation rotation shaft 1145. Here, the actuation rotation shaft 1145 may be disposed closer to the distal end 1104 than the first rotation shaft 1141 is.

Here, in the end tool 1100 according to an embodiment of the present disclosure, the first rotation shaft 1141, which is a yaw rotation shaft, and the actuation rotation shaft 1145 are provided separately rather than as the same shaft. That is, by forming the first rotation shaft 1141, which is a rotation shaft of the pulley 1111/pulley 1121 that are jaw pulleys and a rotation shaft of a yaw motion, and the actuation rotation shaft 1145, which is a rotation shaft of the second jaw 1102 with respect to the first jaw 1101 and a rotation shaft of an actuation motion, to be spaced apart from each other by a certain distance, a space in which the guide tube 1170 and the blade wire 307 accommodated therein can be gently bent may be secured. The actuation rotation shaft 1145 will be described in detail later.

The pulley 1111 functions as an end tool first jaw pulley, and the pulley 1121 functions as an end tool second jaw pulley. The pulley 1111 may also be referred to as a first jaw pulley, and the pulley 1121 may also be referred to as a second jaw pulley, and these two components may also be referred to collectively as an end tool jaw pulley or simply a jaw pulley.

The pulley 1111 and the pulley 1121, which are end tool jaw pulleys, are formed to face each other, and are formed to be rotatable independently of each other around the first rotation shaft 1141 which is an end tool jaw pulley rotation shaft. In this case, the pulley 1111 and pulley 1121 are formed to be spaced apart by a certain distance, and a blade assembly accommodation part may be accommodated therebetween. In addition, at least a portion of a blade assembly to be described later may be disposed in the blade assembly accommodation part. In other words, the blade assembly including the guide tube 1170 may be disposed between the pulley 1111 and the pulley 1121.

Here, since the pulley 1111 is connected to the first jaw 1101, when the pulley 1111 rotates around the first rotation shaft 1141, the first jaw 1101 may also rotate around the first rotation shaft 1141 together with the pulley 1111.

Meanwhile, since the pulley 1121 is connected to the second jaw 1102, when the pulley 1121 rotates around the first rotation shaft 1141, the second jaw 1102 connected to the pulley 1121 may rotate around the first rotation shaft 1141.

In addition, a yaw motion and an actuation motion of the end tool 1100 are performed in response to the rotation of the pulley 1111 and the pulley 1121. That is, when the pulley 1111 and the pulley 1121 rotate in the same direction around the first rotation shaft 1141, the yaw motion is performed as the first jaw 1101 and the second jaw 1102 rotate with the first rotation shaft 1141 as the center of rotation. Meanwhile, when the pulley 1111 and the pulley 1121 rotate in opposite directions around the first rotation shaft 1141, the actuation motion is performed as the first jaw 1101 and the second jaw 1102 rotate around the actuation rotation shaft 1145.

The pulley 1113 and the pulley 1114 function as end tool first jaw pitch main pulleys, and the pulley 1123 and the pulley 1124 function as end tool second jaw pitch main pulleys, and these two components may collectively be referred to as end tool jaw pitch main pulleys.

The pulley 1115 and the pulley 1116 function as end tool first jaw pitch sub-pulleys, and the pulley 1125 and the pulley 1126 function as end tool second jaw pitch sub-pulleys, and these two components collectively may be referred to as end tool jaw pitch sub-pulleys.

Hereinafter, components associated with the rotation of the pulley 1111 will be described.

The pulley 1113 and the pulley 1114 function as end tool first jaw pitch main pulleys. That is, the pulley 1113 and the pulley 1114 function as main rotation pulleys for a pitch motion of the first jaw 1101. Here, the wire 301, which is a first jaw wire, is wound around the pulley 1113, and the wire 305, which is a first jaw wire, is wound around the pulley 1114.

The pulley 1115 and the pulley 1116 function as end tool first jaw pitch sub-pulleys. That is, the pulley 1115 and the pulley 1116 function as sub-rotation pulleys for a pitch motion of the first jaw 1101. Here, the wire 301, which is a first jaw wire, is wound around the pulley 1115, and the wire 305, which is a first jaw wire, is wound around the pulley 1116.

Here, the pulley 1113 and the pulley 1114 are disposed on one side of the pulley 1111 to face each other. Here, the pulley 1113 and the pulley 1114 are formed to be rotatable independently of each other around the third rotation shaft 1143 that is an end tool pitch rotation shaft. In addition, the pulley 1115 and the pulley 1116 are disposed on one side of the pulley 1113 and one side of the pulley 1114, respectively, to face each other. Here, the pulley 1115 and the pulley 1116 are formed to be rotatable independently of each other around the fourth rotation shaft 1144 that is an end tool pitch auxiliary rotation shaft. Here, in the drawings, it is illustrated that the pulley 1113, the pulley 1115, the pulley 1114, and the pulley 1116 are all formed to be rotatable around a Y-axis direction, but the concept of the present disclosure is not limited thereto, and the rotation axes of the respective pulleys may be formed in various directions according to configurations thereof.

The wire 301, which is a first jaw wire, is sequentially wound to make contact with at least portions of the pulley 1115, the pulley 1113, and the pulley 1111. In addition, the wire 305 connected to the wire 301 by the fastening member 323 is sequentially wound to make contact with at least portions of the pulley 1111, the first wire guide part 1168a of the end tool hub 1160, the pulley 1114, and the pulley 1116.

In other words, the wire 301 and the wire 305, which are the first jaw wire, are sequentially wound to make contact with at least portions of the pulley 1115, the pulley 1113, the pulley 1111, the first wire guide part 1168a of the end tool hub 1160, the pulley 1114, and the pulley 1116, and the wire 301 and the wire 305 formed to move along the above pulleys while rotating the above pulleys.

Accordingly, when the wire 301 is pulled in the direction of an arrow 301 of FIG. 13, the fastening member 323 to which the wire 301 is coupled and the pulley 1111 coupled to the fastening member 323 are rotated in a counterclockwise direction. On the contrary, when the wire 305 is pulled in the direction of an arrow 305 of FIG. 13, the fastening member 323 to which the wire 305 is coupled and the pulley 1111 coupled to the fastening member 323 are rotated in a clockwise direction in FIG. 13.

Next, components associated with the rotation of the pulley 1121 will be described.

The pulley 1123 and the pulley 1124 function as end tool second jaw pitch main pulleys. That is, the pulley 1123 and the pulley 1124 function as main rotation pulleys for a pitch motion of the second jaw 1102. Here, the wire 306, which is a second jaw wire, is wound around the pulley 1123, and the wire 302, which is a second jaw wire, is wound around the pulley 1124.

The pulley 1125 and the pulley 1126 function as end tool second jaw pitch sub-pulleys. That is, the pulley 1125 and the pulley 1126 function as sub-rotation pulleys for a pitch motion of the second jaw 1102. Here, the wire 306, which is a second jaw wire, is wound around the pulley 1125, and the wire 302, which is a second jaw wire, is wound around the pulley 1126.

Here, the pulley 1123 and the pulley 1124 are disposed on one side of the pulley 1121 to face each other. Here, the pulley 1123 and the pulley 1124 are formed to be rotatable independently of each other around the third rotation shaft 1143 that is an end tool pitch rotation shaft. In addition, the pulley 1125 and the pulley 1126 are disposed on one side of the pulley 1123 and one side of the pulley 1124, respectively, to face each other. Here, the pulley 1125 and the pulley 1126 are formed to be rotatable independently of each other around the fourth rotation shaft 1144 that is an end tool pitch auxiliary rotation shaft. Here, in the drawings, it is illustrated that all of the pulley 1123, the pulley 1125, the pulley 1124, and the pulley 1126 are formed to be rotatable around the Y-axis direction, but the concept of the present disclosure is not limited thereto, and the rotating axes of the respective pulleys may be formed in various directions according to configurations thereof.

The wire 306, which is a second jaw wire, is sequentially wound to make contact with at least portions of the pulley 1125, the pulley 1123, and the pulley 1121. In addition, the wire 302 connected to the wire 306 by the fastening member 326 is sequentially wound to make contact with at least portions of the pulley 1121, the second wire guide part 1168b of the end tool hub 1160, the pulley 1124, and the pulley 1126.

In other words, the wire 306 and the wire 302, which are the second jaw wire, are sequentially wound to make contact with at least portions of the pulley 1125, the pulley 1123, the pulley 1121, the second wire guide part 1168b of the end tool hub 1160, the pulley 1124, and the pulley 1126, and the wire 306 and the wire 302 are formed to move along the above pulleys while rotating the above pulleys.

Accordingly, when the wire 306 is pulled in the direction of an arrow 306 of FIG. 13, the fastening member 326 to which the wire 306 is coupled and the pulley 1121 coupled to the fastening member 326 are rotated in the clockwise direction in FIG. 13. On the contrary, when the wire 302 is pulled toward an arrow 302 of FIG. 13, the fastening member 326 coupled to the wire 302 and the pulley 1121 coupled to the fastening member 326 may rotate in the counterclockwise direction in FIG. 13.

Hereinafter, a pitch motion of the present disclosure will be described in more detail.

Meanwhile, when the wire 301 is pulled in the direction of the arrow 301 of FIG. 13, and simultaneously, the wire 305 is pulled in the direction of the arrow 305 of FIG. 13 (that is, when both strands of the first jaw wire are pulled), as shown in FIG. 12, since the wires 301 and 305 are wound around lower portions of the pulley 1113 and the pulley 1114 rotatable around the third rotation shaft 1143, which is an end tool pitch rotation shaft, the pulley 1111 to which the wires 301 and 305 are fixedly coupled and the end tool hub 1160 to which the pulley 1111 is coupled rotate as a whole in the counterclockwise direction around the third rotation shaft 1143, and as a result, the end tool 1100 may rotate downward to perform the pitch motion. At this time, since the second jaw 1102 and the wires 302 and 306 fixedly coupled thereto are wound around upper portions of the pulley 1123 and the pulley 1124 rotatable around the third rotation shaft 1143, the wires 302 and 306 are released in the opposite directions of the arrows 302 and 306, respectively.

On the contrary, when the wire 302 is pulled in the direction of the arrow 302 of FIG. 13, and simultaneously, the wire 306 is pulled in the direction of the arrow 306 of FIG. 13, as shown in FIG. 12, since the wires 302 and 306 are wound around the upper portions of the pulley 1123 and the pulley 1124 rotatable around the third rotation shaft 1143, which is an end tool pitch rotation shaft, the pulley 1121 to which the wires 302 and 306 are fixedly coupled and the end tool hub 1160 to which the pulley 1121 is coupled rotate as a whole in the clockwise direction around the third rotation shaft 1143, and as a result, the end tool 1100 may rotate upward to perform the pitch motion. At this time, since the first jaw 1101 and the wires 301 and 305 fixedly coupled thereto are wound around lower portions of the pulley 1113 and the pulley 1114 rotatable around the third rotation shaft 1143, the wires 302 and 306 are moved in the opposite directions of the arrows 301 and 305, respectively.

Meanwhile, the end tool hub 1160 of the end tool 1100 of the surgical instrument 10 of the present disclosure may further include the first pitch pulley part 1163a and the second pitch pulley part 1163b serving as end tool pitch pulleys, the manipulation part 200 may further include the pulley 231 and a pulley 232, which are manipulation part pitch pulleys, and the power transmission part 300 may further include the wire 303 and the wire 304 which are pitch wires.

In detail, the end tool hub 1160 including the first pitch pulley part 1163a and the second pitch pulley part 1163b may be formed to be rotatable around the third rotation shaft 1143 that is an end tool pitch rotation shaft. In addition, the wires 303 and 304 may serve to connect the first and second pitch pulley parts 1163a and 1163b of the end tool 1100 to the pulleys 231 and 232 of the manipulation part 200.

Thus, when the pulleys 231 and 232 of the manipulation part 200 rotate, the rotation of the pulleys 231 and 232 is transmitted to the end tool hub 1160 of the end tool 1100 through the wires 303 and 304, causing the end tool hub 1160 to rotate as well, and as a result, the end tool 1100 performs a pitch motion while rotating.

That is, the surgical instrument 10 according to an embodiment of the present disclosure includes the first and second pitch pulley parts 1163a and 1163b of the end tool 1100, the pulleys 231 and 232 of the manipulation part 200, and the wires 303 and 304 of the power transmission part 300 in order to transmit driving force for a pitch motion, and thus, the driving force for the pitch motion of the manipulation part 200 is more completely transmitted to the end tool 1100, thereby improving operation reliability.

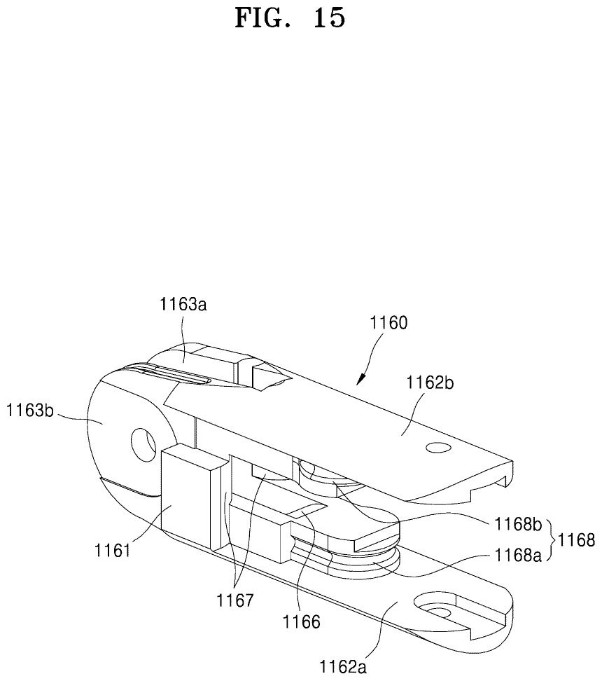

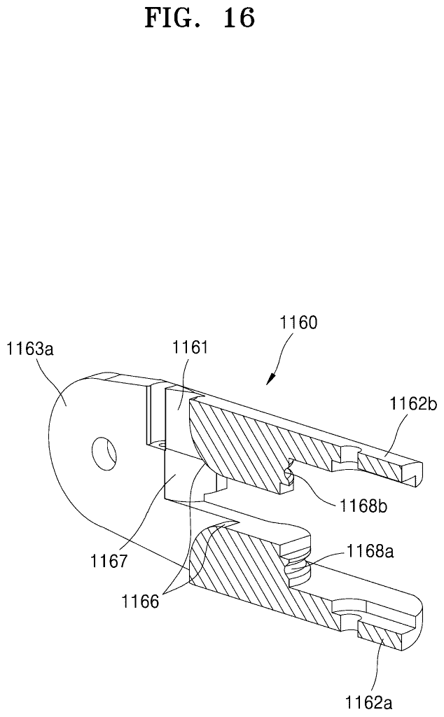

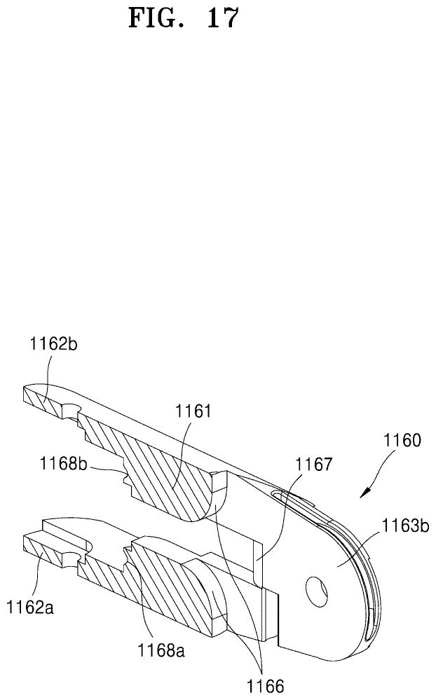

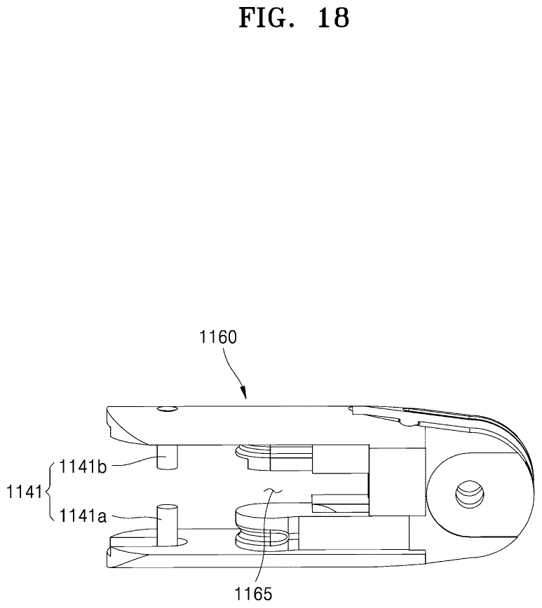

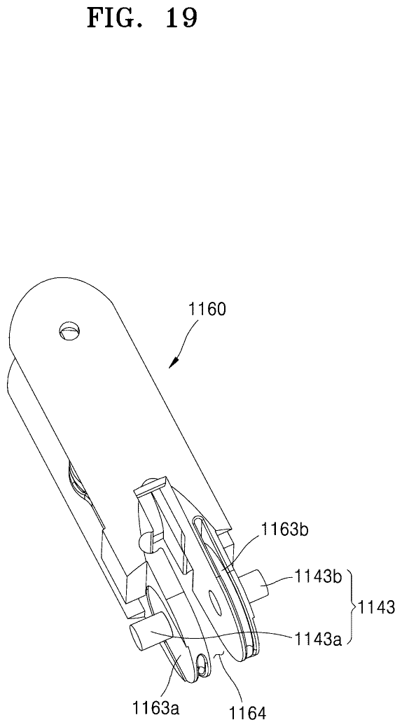

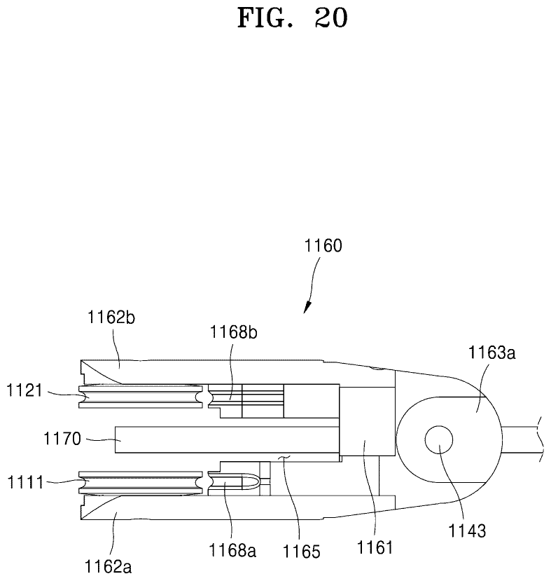

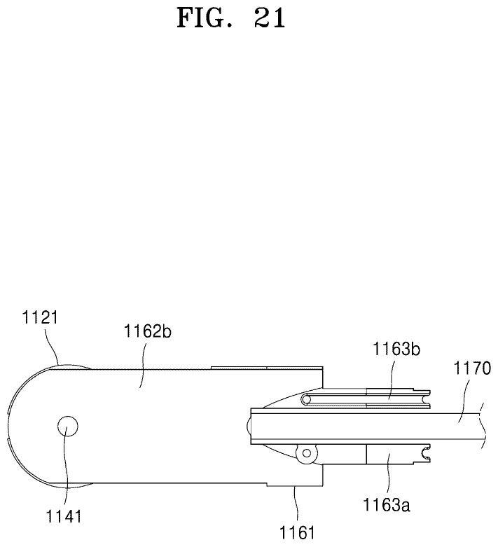

FIG. 15 is a perspective view illustrating the end tool hub of the surgical instrument of FIG. 7. FIGS. 16 and 17 are cut-away perspective views of the end tool hub of FIG. 15. FIGS. 18 and 19 are perspective views illustrating the end tool hub of FIG. 15. FIG. 20 is a side view illustrating the end tool hub of FIG. 15 and the guide tube. FIG. 21 is a plan view illustrating the end tool hub of FIG. 15 and the guide tube.

Referring to FIGS. 15 to 21, the end tool hub 1160 includes a body part 1161, a first jaw pulley coupling part 1162a, a second jaw pulley coupling part 1162b, the first pitch pulley part 1163a, the second pitch pulley part 1163b, a pitch slit 1164, a yaw slit 1165, ta he pitch round part 1166, a yaw round part 1167, and the wire guide part 1168. In addition, the wire guide part 1168 includes the first wire guide part 1168a and the second wire guide part 1168b.

The first jaw pulley coupling part 1162a and the second jaw pulley coupling part 1162b may be formed in the end tool hub 1160 at the distal end side. Here, the first jaw pulley coupling part 1162a and the second jaw pulley coupling part 1162b are formed to face each other, and the pulley 1111 and the pulley 1121 are accommodated therein. Here, the first jaw pulley coupling part 1162a and the second jaw pulley coupling part 1162b may be formed to be approximately parallel to a plane perpendicular to the first rotation shaft 1141 that is a yaw rotation shaft.

The first jaw pulley coupling part 1162a and the second jaw pulley coupling part 1162b are connected by the body part 1161. That is, the first jaw pulley coupling part 1162a and the second jaw pulley coupling part 1162b, which are parallel to each other, are coupled by the body part 1161 formed in a direction approximately perpendicular to the first jaw pulley coupling part 1162a and the second jaw pulley coupling part 1162b, so that the first jaw pulley coupling part 1162a, the second jaw pulley coupling part 1162b, and the body part 1161 form an approximately U-shape, in which the pulley 1111 and the pulley 1121 are accommodated.

In other words, it may be said that the first jaw pulley coupling part 1162a and the second jaw pulley coupling part 1162b are formed to extend in the X-axis direction from the body part 1161.

Here, the pulley 1111, which is a first jaw pulley, is disposed close to the first jaw pulley coupling part 1162a of the end tool hub 1160, and the pulley 1121, which is a second jaw pulley, is disposed close to the second jaw pulley coupling part 1162b of the end tool hub 1160, and thus the yaw slit 1165 may be formed between the first jaw pulley coupling part 1162a and the second jaw pulley coupling part 1162b. In addition, at least a portion of the blade assembly to be described later may be disposed in the yaw slit 1165. In other words, it may be said that at least a portion of the guide tube 1170 of the blade assembly may be disposed between the first jaw pulley coupling part 1162a and the second jaw pulley coupling part 1162b. As such, by disposing the blade assembly including the guide tube 1170 between the pulley 1111, which is a first jaw pulley, and the pulley 1121, which is a second jaw pulley, the end tool 1100 is able to perform the cutting motion using the blade 1175 in addition to the pitch and yaw motions. This will be described in more detail later.

Meanwhile, a through hole is formed in the first jaw pulley coupling part 1162a such that the first rotation shaft 1141 passes through the first jaw pulley coupling part 1162a and the pulley 1111 and axially couples the first jaw pulley coupling part 1162a and the pulley 1111. In addition, a through hole is formed in the second jaw pulley coupling part 1162b such that the first rotation shaft 1141 passes through the second jaw pulley coupling part 1162b and the pulley 1121 and axially couples the second jaw pulley coupling part 1162b and the pulley 1121.

Here, as described above, the first rotation shaft 1141, which is a yaw rotation shaft, may be formed by being divided into two parts of the first sub-shaft 1141a and the second sub-shaft 1141b, and the guide tube 1170 may pass between the first sub-shaft 1141a and the second sub-shaft 1141b of the first rotation shaft 1141.

In addition, the yaw slit 1165 may be formed between the first jaw pulley coupling part 1162a and the second jaw pulley coupling part 1162b. Since the yaw slit 1165 is formed in the end tool hub 1160 as described above, the guide tube 1170 may pass through the inside of the end tool hub 1160.

In other words, the first rotation shaft 1141 is vertically separated into two parts without passing through the end tool hub 1160, and the yaw slit 1165 may be formed on a plane perpendicular to the first rotation shaft 1141 in the vicinity of the first rotation shaft 1141. Accordingly, the guide tube 1170 is movable (i.e., movable left and right) in the yaw slit 1165 while passing through the vicinity of the first rotation shaft 1141.

Meanwhile, the yaw round part 1167 may be further formed in the body part 1161. The yaw round part 1167 may be formed to be rounded so as to have a predetermined curvature. In detail, when viewed from a plane perpendicular to the first rotation shaft 1141 that is a yaw rotation shaft, the yaw round part 1167 may be formed to be rounded so as to have a predetermined curvature. For example, the yaw round part 1167 may be formed in a fan shape, and may be formed along a path in which the guide tube 1170 is bent on an XY plane. The yaw round part 1167 as described above may serve to guide the path of the guide tube 1170 when the end tool 1100 yaw-rotates.

The wire guide part 1168, which guides a path of the wire passing through the inside of the end tool hub 1160, is formed at one side of the body part 1161. Here, the wire guide part 1168 includes the first wire guide part 1168a and the second wire guide part 1168b. Here, the first wire guide part 1168a may be formed on an inner side surface of the first jaw pulley coupling part 1162a. In addition, the second wire guide part 1168b may be formed on an inner side surface of the second jaw pulley coupling part 1162b.

Here, the wire guide part 1168 may be formed in a cylindrical shape with a cross section that is approximately semi-circular. In addition, the semi-circular portion may be disposed to protrude toward the pulley 1111 and the pulley 1121. In other words, it may be said that the wire guide part 1168 is formed to protrude toward a space formed by the first jaw pulley coupling part 1162a, the second jaw pulley coupling part 1162b, and the body part 1161. In other words, it may be said that, in the wire guide part 1168, a region adjacent to the first jaw pulley coupling part 1162a and the second jaw pulley coupling part 1162b is formed to have a cross section that is curved with a predetermined curvature.

Alternatively, in other words, it may be also said that the wire guide part 1168 functions as a kind of pulley member, which guides the paths of the wire 305 and the wire 302 by winding the wire 305 and the wire 302 around an outer circumferential surface thereof. However, here, the wire guide part 1168 is not a member that rotates around a certain shaft as the original meaning pulley does, and it may be said that the wire guide part 1168 is formed to be fixed as a portion of the end tool hub 1160 and performs some similar functions of a pulley by winding a wire therearound.

Here, the wire guide part 1168 is illustrated in the drawing as being formed in a cylindrical shape with a cross section that is approximately semi-circular. That is, at least a portion of the cross section of the wire guide part 1168 on the XY plane is illustrated as having a certain arc shape. However, the concept of the present disclosure is not limited thereto, and the cross section may have a predetermined curvature like an oval or a parabola, or a corner of a polygonal column is rounded to a certain degree, so that the cross section may have various shapes and sizes suitable for guiding the paths of the wire 305 and the wire 302.

Here, a guide groove for guiding the paths of the wire 305 and the wire 302 well may be further formed in a portion of the wire guide part 1168, which is in contact with the wire 305 and the wire 302. The guide groove may be formed in the form of a groove recessed to a certain degree from a protruding surface of the wire guide part 1168.

Here, although the guide groove is illustrated in the drawing as being formed in the entire arc surface of the wire guide part 1168, the concept of the present disclosure is not limited thereto, and the guide groove may be formed only in a portion of the arc surface of the wire guide part 1168 as necessary.

As described above, by further forming the guide groove in the wire guide part 1168, unnecessary friction between the wires is reduced, so that durability of the wires may be improved.

The first pitch pulley part 1163a and the second pitch pulley part 1163b, which serve as end tool pitch pulleys, may be formed on the end tool hub 1160 at the proximal end side. Here, the first pitch pulley part 1163a and the second pitch pulley part 1163b may be formed to face each other. Here, the first pitch pulley part 1163a and the second pitch pulley part 1163b may be formed to be approximately parallel to a plane perpendicular to the third rotation shaft 1143, which is a pitch rotation shaft.

In detail, one end portion of the end tool hub 1160 is formed in a disk shape similar to a pulley, and grooves around which a wire may be wound may be formed on an outer circumferential surface of the one end portion, thereby forming the first pitch pulley part 1163a and the second pitch pulley part 1163b The wire 303 and the wire 304 described above are coupled to the first pitch pulley part 1163a and the second pitch pulley part 1163b, which serve as end tool pitch pulleys, and a pitch motion is performed while the end tool hub 1160 rotates around the third rotation shaft 1143.

Meanwhile, although not shown in the drawings, the pitch pulley may be formed as a separate member from the end tool hub 1160 and coupled to the end tool hub 1160.

The first pitch pulley part 1163a and the second pitch pulley part 1163b may be connected by the body part 1161. That is, the first pitch pulley part 1163a and the second pitch pulley part 1163b, which are parallel to each other, are coupled by the body part 1161 formed in a direction approximately perpendicular to the first pitch pulley part 1163a and the second pitch pulley part 1163b, and thus the first pitch pulley part 1163a, the second pitch pulley part 1163b, and the body part 1161 may form an approximately U-shape.

In other words, it may be said that the first pitch pulley part 1163a and the second pitch pulley part 1163b are formed to extend from the body part 1161 in the X-axis direction.

Meanwhile, a through hole is formed in the first pitch pulley part 1163a so that the third rotation shaft 1143 may pass through the first pitch pulley part 1163a. In addition, a through hole is formed in the second pitch pulley part 1163b so that the third rotation shaft 1143 may pass through the second pitch pulley part 1163b.

In this case, as described above, the third rotation shaft 1143, which is a pitch rotation shaft, may be formed by being divided into two parts of the first sub-shaft 1143a and the second sub-shaft 1143b, and the guide tube 1170 may pass between the first sub-shaft 1143a and the second sub-shaft 1143b of the third rotation shaft 1143.

The pitch slit 1164 may be formed between the first pitch pulley part 1163a and the second pitch pulley part 1163b. Since the pitch slit 1164 is formed in the end tool hub 1160 as described above, the guide tube 1170 may pass through the inside of the end tool hub 1160.

In other words, the third rotation shaft 1143 is horizontally separated into two parts without passing through the end tool hub 1160, and the pitch slit 1164 may be formed on a plane perpendicular to the third rotation shaft 1143 in the vicinity of the third rotation shaft 1143. Accordingly, the guide tube 1170 is movable (movable up and down) in the pitch slit 1164 while passing through the vicinity of the third rotation shaft 1143.

Meanwhile, the pitch round part 1166 may be further formed in the body part 1161. The pitch round part 1166 may be formed to be rounded to have a predetermined curvature. In detail, when viewed from a plane perpendicular to the third rotation shaft 1143, which is a pitch rotation shaft, the pitch round part 1166 may be formed to be rounded to have a predetermined curvature. For example, the pitch round part 1166 may be formed in a fan shape, and formed along a path in which the guide tube 1170 is bent on the XZ plane. The pitch round part 1166 as described above may serve to guide the path of the guide tube 1170 when the end tool 1100 pitch-rotates.

Here, the pitch slit 1164 and the yaw slit 1165 may be formed to be connected to each other. Accordingly, the guide tube 1170 and the blade wire 307 located therein may be disposed to completely pass through the inside of the end tool hub 1160. In addition, the blade 1175 coupled to one end portion of the blade wire 307 may linearly reciprocate inside the first jaw 1101 and the second jaw 1102.

As described above, since the blade wire 307 and the guide tube 1170 need to be connected to the blade 1175 through the end tool hub 1160, and a space in which the blade wire 307 and the guide tube 1170 can be bent in the end tool hub 1160 is necessary, in the present disclosure, 1) spaces, through which the blade wire 307/the guide tube 1170 can pass and simultaneously are bendable, that is, the pitch slit 1164 and the yaw slit 1165, are formed in the end tool hub 1160, 2) the rotation shafts are formed by being divided into two parts, and 3) the pitch round part 1166 and the yaw round part 1167 are additionally formed to guide the bending of the blade wire 307/the guide tube 1170.

Hereinafter, the role and function of the wire guide part 1168 will be described in more detail.

The wire guide part 1168 may be in contact with the wire 305 and the wire 302 and may change the arrangement path of the wire 305 and the wire 302 to a certain degree to serve to increase a rotation radius of each of the first jaw 1101 and the second jaw 1102.

That is, when the auxiliary pulleys are not disposed, each of the pulley 1111, which is a first jaw pulley, and the pulley 1121, which is a second jaw pulley, may rotate up to a right angle, but in an embodiment of the present disclosure, by additionally providing the wire guide part 1168 in the end tool hub 1160, the maximum rotation angle of each pulley may be increased.

This enables a motion in which two jaws of the end tool 1100 have to be spread apart for an actuation motion in a state in which the two jaws are yaw-rotated together by 90°. In other words, the range of yaw rotation in which an actuation motion is possible may be increased through the configuration of the wire guide part 1168 of the end tool hub 1160. In other words, the range of yaw rotation in which an actuation motion is possible may be increased through the configuration of the wire guide part 1168 of the end tool hub 1160.

Furthermore, by forming the wire guide part 1168 in the end tool hub 1160, which already exists, without adding a separate structure such as an auxiliary pulley, the range of rotation may be increased without adding a component and a manufacturing process.

As described above, since there is no need to additionally dispose a separate structure for increasing the rotation angle, the number of components is decreased and the manufacturing process is simplified, and also the length of the end tool is shortened as much as the size of the auxiliary pulley, so that the length of the end tool is shortened during a pitch motion. Accordingly, a surgical motion may be more easily performed in a narrow space.

This will be described later in more detail.

In the end tool 1100 of the surgical instrument according to an embodiment of the present disclosure, the arrangement path of the wires may be changed without a separate structure by forming the wire guide part 1168 capable of changing the path of the wire on an inner side wall of the end tool hub 1160. As described above, as the arrangement path of the wire 305 and the wire 302 is changed to a certain degree by forming the wire guide part 1168 in the end tool hub 1160, a tangential direction of the wire 305 and the wire 302 is changed, and accordingly, rotation angles of the fastening member 323 and the fastening member 326 that couple respective wires and pulleys may be increased.

That is, the fastening member 326 that couples the wire 302 and the pulley 1121 is rotatable until being located on a common internal tangent of the pulley 1121 and the wire guide part 1168. Similarly, the fastening member (see 323 of FIG. 12) that couples the wire 305 and the pulley 1111 is rotatable until being located on a common internal tangent of the pulley 1111 and the wire guide part 1168, so that a rotation angle of the fastening member (see 323 of FIG. 12) may be increased.

In other words, the wire 301 and the wire 305 wound around the pulley 1111 by the wire guide part 1168 are disposed on one side with respect to a plane perpendicular to the Y-axis and passing through the X-axis. Simultaneously, the wire 302 and the wire 306 wound around the pulley 1121 by the wire guide part 1168 are disposed on the other side with respect to the plane perpendicular to the Y-axis and passing through the X-axis.

In other words, the pulley 1113 and the pulley 1114 are disposed at one side with respect to the plane perpendicular to the Y-axis and passing through the X-axis, and the pulley 1123 and the pulley 1124 are disposed at the other side with respect to the plane perpendicular to the Y-axis and passing through the X-axis.

In other words, the wire 305 is located on the internal tangent of the pulley 1111 and the wire guide part 1168, and a rotation angle of the pulley 1111 is increased due to the wire guide part 1168. In addition, the wire 302 is located on the internal tangent of the pulley 1121 and the wire guide part 1168, and the rotation angle of the pulley 1121 is increased due to the wire guide part 1168.

In the present embodiment in which an auxiliary pulley is not formed and the wire guide part 1168 capable of changing the path of a wire is formed on the inner side wall of the end tool hub 1160, the length of the end tool of the surgical instrument may be shortened as compared to the surgical instrument of the embodiment in which a separate auxiliary pulley is formed. Since the length of the end tool is reduced as described above, a surgical operator may easily manipulate a surgical instrument, and side effects of surgery may be reduced when the surgery is performed in a narrow surgical space in the human body.

According to the present disclosure as described above, the rotation radii of the pulley 1111, which is a first jaw pulley, and the pulley 1121, which is a second jaw pulley, increase, so that a yaw motion range in which a normal opening/closing actuation motion and a normal cutting motion can be performed may be increased.

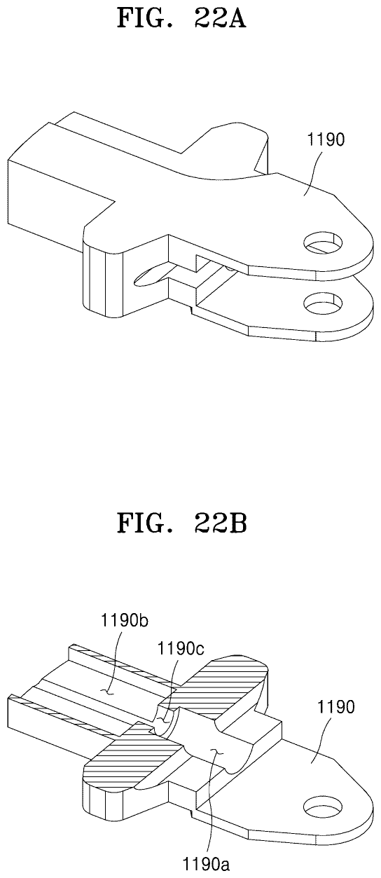

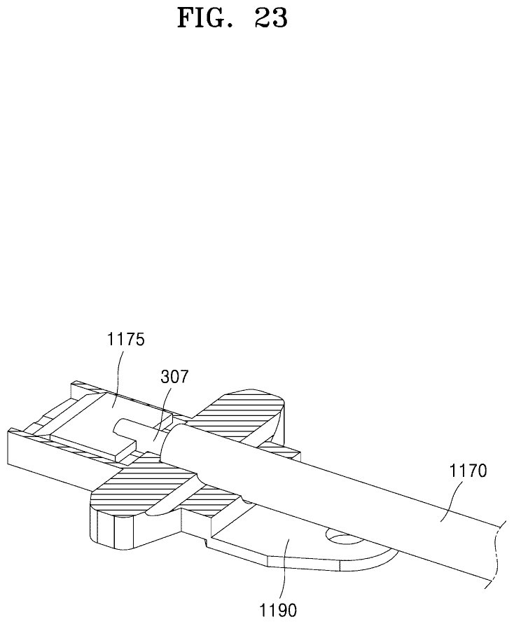

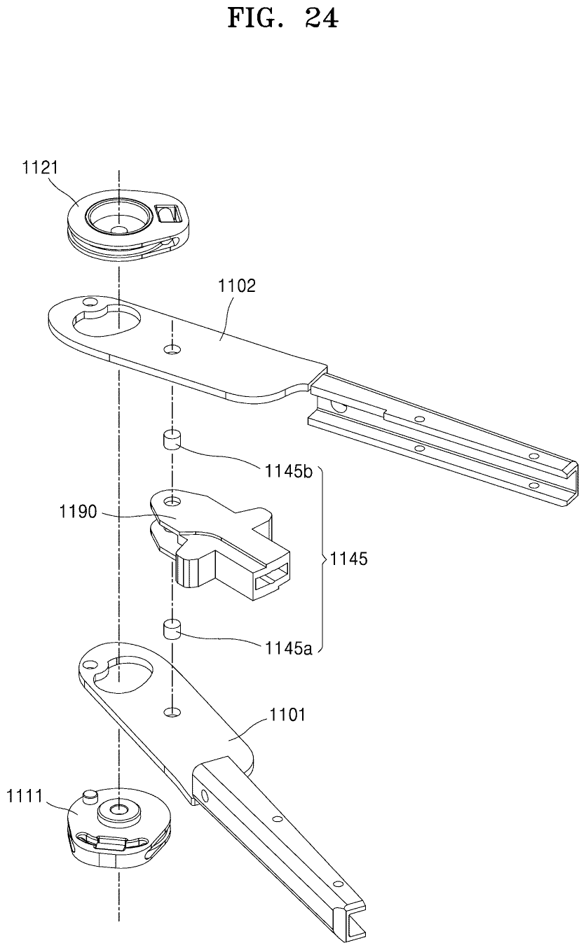

FIG. 22 is a set of perspective and cut-away perspective views illustrating an actuation hub of the surgical instrument of FIG. 7. FIG. 23 is a view illustrating a state in which the guide tube, the blade wire, and the blade are mounted on the actuation hub illustrated in the cut-away perspective view of FIG. 22. FIG. 24 is an exploded perspective view illustrating the end tool of the surgical instrument of FIG. 7.

Referring to FIGS. 22 to 24, an actuation hub 1190 may be formed in the form of a box having a hollow therein. In addition, the actuation hub 1190 is coupled to each of the first jaw 1101 and the second jaw 1102. In detail, the actuation hub 1190 is axially coupled to the first jaw 1101 by a first actuation rotation shaft 1145a. In addition, the actuation hub 1190 is axially coupled to the second jaw 1102 by a second actuation rotation shaft 1145b. In this case, the first actuation rotation shaft 1145a and the second actuation rotation shaft 1145b may be disposed on the same line in a Z-axis direction.

In addition, a tube seating part 1190a may be formed inside the actuation hub 1190, and one end portion of the guide tube 1170 may be fixedly coupled to the tube seating part 1190a.

Meanwhile, a blade accommodation part 1190b may be formed inside the actuation hub 1190, and the blade 1175 may be accommodated in the blade accommodation part 1190b.

In addition, a wire through-hole 1190c may be formed between the tube seating part 1190a and the blade accommodation part 1190b inside the actuation hub 1190.

That is, the tube seating part 1190a, the wire through-hole 1190c, and the blade accommodation part 1190b are sequentially formed inside the actuation hub 1190, and the blade wire 307 may pass through the inside of the actuation hub 1190 to be connected to the blade 1175.

As described above, by providing the actuation hub 1190 to which the guide tube 1170 is coupled between the first jaw 1101 and the second jaw 1102, the guide tube 1170 may not be curved, or the angle at which the guide tube 1170 is curved may be reduced, even when the first jaw 1101 or the second jaw 1102 rotates around the first rotation shaft 1141 or the actuation rotation shaft 1145.

In detail, in a case in which the guide tube 1170 is directly coupled to the first jaw 1101 or the second jaw 1102, when the first jaw 1101 or the second jaw 1102 rotates, one end portion of the guide tube 1170 also rotates together with the first jaw 1101 or the second jaw 1102, causing the guide tube 1170 to be curved.

On the other hand, in a case in which the guide tube 1170 is coupled to the actuation hub 1190, which is independent of the rotation of the jaw 1103, as in the present embodiment, even when the first jaw 1101 or the second jaw 1102 rotates, the guide tube 1170 may not be curved, or the angle at which the guide tube 1170 is curved may be reduced even when the guide tube 1170 is curved.

That is, by changing the direct connection between the guide tube 1170 and the jaw 1103 by the actuation hub 1190 to an indirect connection, the degree to which the guide tube 1170 is curved by the rotation of the jaw 1103 may be reduced.

Subsequently, referring to FIGS. 7 to 30 and the like, the end tool 1100 according to an embodiment of the present disclosure may include the first jaw 1101, the second jaw 1102, a first electrode 1151, a second electrode 1152, the guide tube 1170, and the blade 1175 in order to perform cauterizing and cutting motions. However, the position and number of electrodes are not limited thereto, and the end tool 1100 may include only the first electrode 1151 formed on the first jaw 1101, or only the second electrode 1152 formed on the second jaw 1102.

Here, components related to the driving of the blade, such as the guide tube 1170 and the blade 1175, may be collectively referred to as a blade assembly. In an embodiment of the present disclosure, by disposing the blade assembly including the guide tube 1170 and the blade 1175 between the pulley 1111, which is a first jaw pulley, and the pulley 1121, which a second jaw pulley, the end tool 1100 is able to perform the cutting motion using the blade 1175 in addition to the pitch and yaw motions. This will be described in more detail.

As described above, the first jaw 1101 is connected to the first jaw pulley 1111 and rotates around the first rotation shaft 1141 together with the first jaw pulley 1111 when the first jaw pulley 1111 rotates around the first rotation shaft 1141.

Meanwhile, the first electrode 1151 may be formed on a surface of the first jaw 1101 facing the second jaw 1102. In addition, the second electrode 1152 may be formed on a surface of the second jaw 1102 facing the first jaw 1101.

At this time, a slit 1151a may be formed in the first electrode 1151, and the blade 1175 may move along the slit 1151a. In addition, a slit 1152a may be formed in the second electrode 1152, and the blade 1175 may move along the slit 1152a.

Meanwhile, although not shown in the drawings, a spacer (not shown) may be formed between the first jaw 1101 and the first electrode 1151, and a spacer (not shown) may be formed between the second jaw 1102 and the second electrode 1152. The spacer (not shown) may include an insulating material such as ceramic. Alternatively, the first jaw 1101 and the second jaw 1102 may themselves be made of a nonconductor such that the first electrode 1151 and the second electrode 1152 may be maintained to be insulated from each other without a separate insulator until the first electrode 1151 and the second electrode 1152 are in contact with each other.

In an embodiment, instead of providing a separate sensor, monitoring and controlling of at least some of current, voltage, resistance, impedance, and temperature may be directly performed by a generator (not shown) which supplies power to the electrodes. Alternatively, if the surgical instrument 10 is a hand-held type device, the processor of the manipulation part 200, which controls power supplied to the electrode, may perform cauterization based on momentary current values corresponding to the power. At this time, the momentary current values are affected by at least some of the voltage, resistance, impedance, and temperature, allowing the processor to predict whether cauterization has been completed without a separate temperature sensor or monitoring device.

An edge portion formed sharply and configured to cut the tissue may be formed in one region of the blade 1175. The tissue disposed between the first jaw 1101 and the second jaw 1102 may be cut as at least a portion of the blade 1175 moves between the distal end 1104 and the proximal end 1105 of the end tool 1100.

Meanwhile, in an embodiment, the electrode may be formed only on one of the first jaw 1101 and the second jaw 1102. For example, the first electrode 1151 may be formed on a surface of the first jaw 1101 facing the second jaw 1102, and the electrode may not be formed on the second jaw 1102. Alternatively, the second electrode 1152 may be formed on a surface of the second jaw 1102 facing the first jaw 1101, and the electrode may not be formed on the first jaw 1101. In this case, a current sensor 451, which will be described later, may be disposed only on the electric wire corresponding to the jaw on which the electrode is formed.

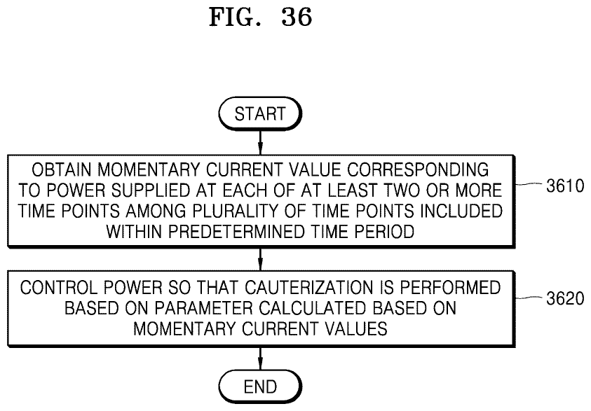

In an embodiment, the electrodes 1151 and 1152 may receive power from the manipulation part 200, may be Joule heated, and may perform cauterization on the tissue. This will be described in more detail later with reference to FIGS. 36 to 40.

Here, the guide tube 1170 and the blade 1175 disposed between the pulley 1111 and the pulley 1121 may be provided in the end tool 1100 of the surgical instrument 10 according to an embodiment of the present disclosure. In addition, by providing the guide tube 1170 and the blade 1175 as described above, a multi-joint/multi-degree-of-freedom surgical instrument capable of pitch/yaw/actuation motions can be implemented to perform both cauterization and cutting. This will be described later in more detail.

Until now, various types of surgical instruments have been developed. Among the various types of surgical instruments for electrocautery, a blood vessel resection device called “Advanced Energy Device” or “Vessel Sealer” has a sensing function added to the existing bipolar cautery method, so that power of different polarities may be supplied to two electrodes, and after denaturing a vessel with the heat generated therefrom for hemostasis, the stanched part may be cut with a blade. At this time, the method involves measuring an impedance of the tissue (or blood vessel) while a current is flowing therethrough to determine whether cauterization is completed, and once the cauterization is completed, the supply of the current is automatically stopped, and the tissue is cut with the blade.

In the case of such a bipolar-type blood vessel resection device, it is essential to have a blade to cut the tissue after cauterization, and the end tool needs to be equipped with a mechanism for facilitating a linear motion of the blade, and thus joint movements such as pitch/yaw movements are not possible in most cases.

On the other hand, in the surgical instrument 10 according to an embodiment, as the electrode is heated in an open-loop manner, the need for a separate sensor to measure the impedance of the tissue can be eliminated, and by heating the electrode through Joule heating, the electrode can be heated without supplying power of different polarities to the two electrodes.

Meanwhile, there have been attempts to implement joint movements using flexible joints with multiple nodes connected in the bipolar-type blood vessel resection device, but in this case, a rotation angle is limited and it is difficult to achieve accurate motion control of the end tool.

On the other hand, in the case of a method that utilizes vibration of ultrasonic waves to perform hemostasis and cutting, it is not feasible to provide joints due to the physical characteristics of ultrasonic waves.

To address these problems, the end tool 1100 of the surgical instrument 10 according to an embodiment of the present disclosure includes the guide tube 1170 disposed between the pulley 1111 and the pulley 1121, and the blade 1175 that moves between a first position and a second position in response to the movement of the blade wire 307 disposed inside the guide tube 1170.

In addition, by providing the guide tube 1170 and the blade 1175 as described above, pitch/yaw/actuation motions may also be performed using a pulley/wire in a bipolar-type surgical instrument for cauterizing and cutting tissue.

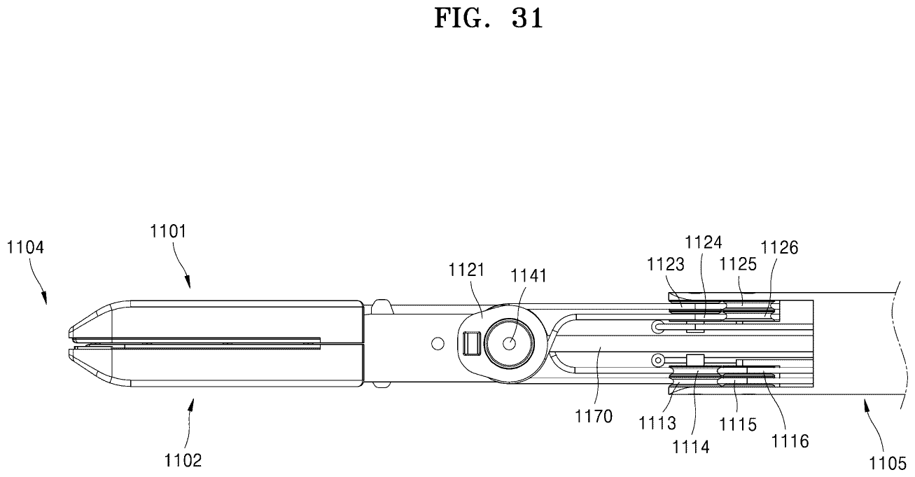

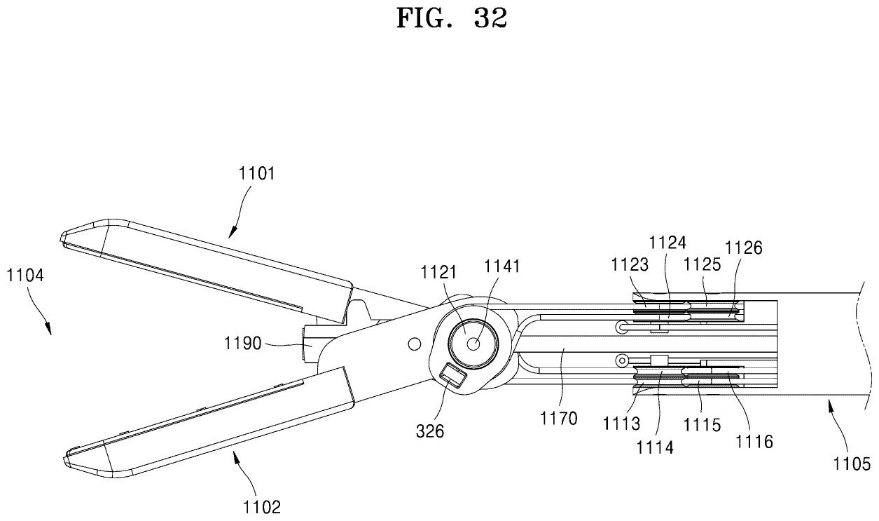

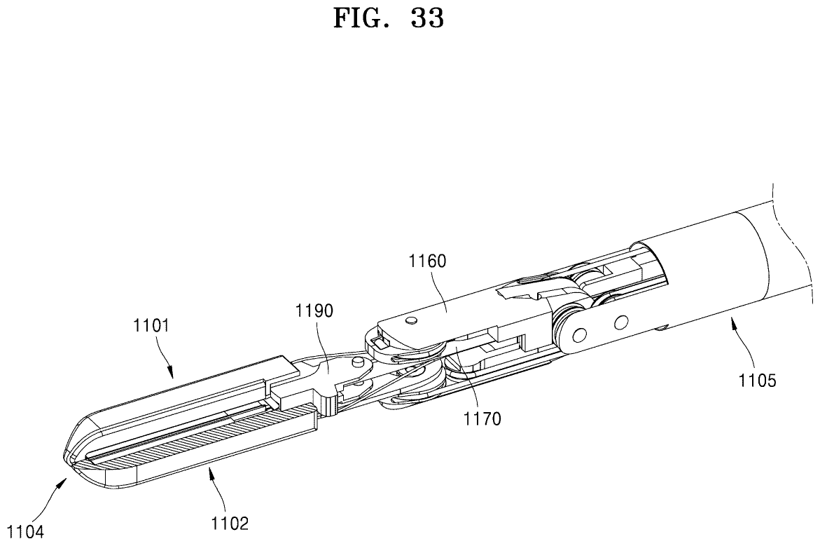

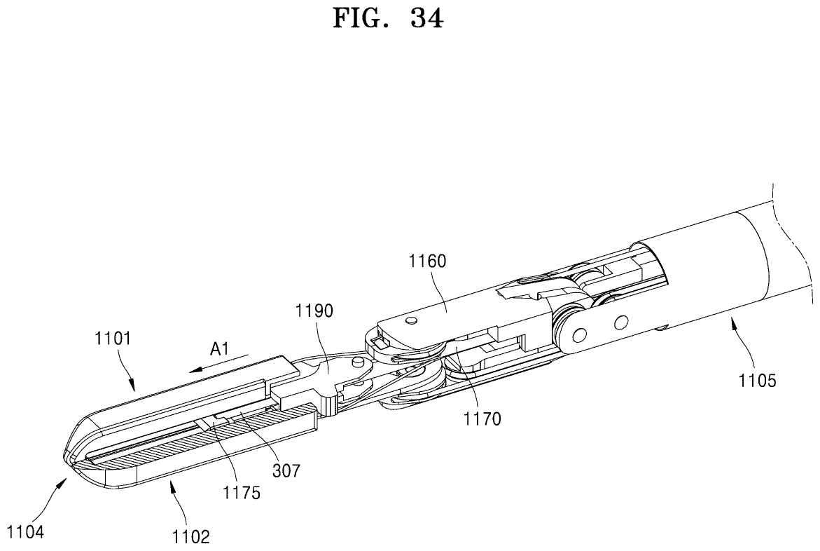

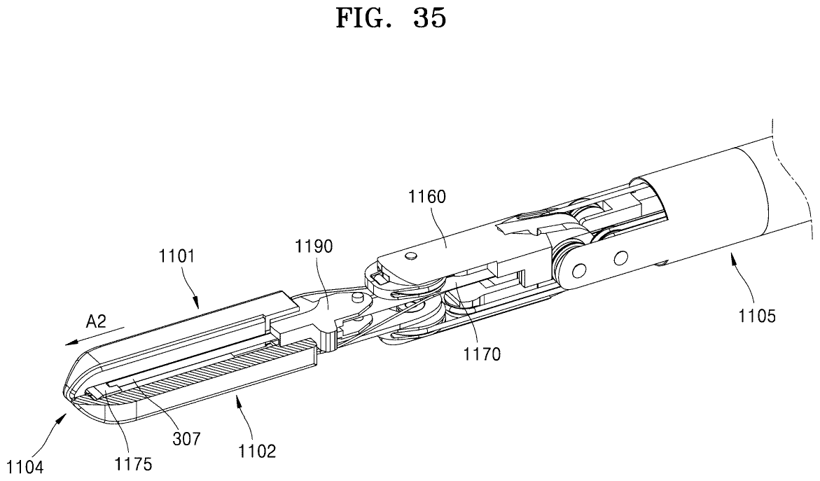

FIG. 31 is a view illustrating a state in which the end tool of the surgical instrument of FIG. 7 is closed, and FIG. 32 is a view illustrating a state in which the end tool of the surgical instrument of FIG. 7 is open. In addition, FIG. 33 is a view illustrating a state in which the blade wire 307 and the blade 1175 are located at a first position, FIG. 34 is a view illustrating a state in which the blade wire 307 and the blade 1175 are located at a second position, and FIG. 35 is a view illustrating a state in which the blade wire 307 and the blade 1175 are located at a third position.

Referring to FIGS. 31 to 35, it may be said that the tissue between the first jaw 1101 and the second jaw 1102 is cut as the cutting motion of FIGS. 33 to 35 is performed in a state in which the first jaw 1101 and the second jaw 1102 are closed as shown in FIG. 31.

Here, the first position illustrated in FIG. 33 may be defined as a state in which the blade 1175 is drawn in toward the proximal end 1105 of the end tool 1100 as much as possible. Alternatively, the first position may be defined as a state in which the blade 1175 is located adjacent to the pulley 1111/pulley 1121.

Meanwhile, the third position illustrated in FIG. 35 may be defined as a state in which the blade 1175 is withdrawn toward the distal end 1104 of the end tool 1100 as much as possible. Alternatively, the third position may be defined as a state in which the blade 1175 is spaced away from the pulley 1111/pulley 1121 as much as possible.

First, as shown in FIG. 32, a tissue to be cut is located between the first jaw 1101 and the second jaw 1102 in a state in which the first jaw 1101 and the second jaw 1102 are opened, and then an actuation motion is performed to close the first jaw 1101 and the second jaw 1102 as shown in FIG. 31.

Next, as shown in FIG. 33, in a state in which the blade wire 307 and the blade 1175 are located at the first position, currents of different polarities are applied to the first electrode 1151 and the second electrode 1152 to cauterize the tissue between the first jaw 1101 and the second jaw 1102. Alternatively, the tissue between the first jaw 1101 and the second jaw 1102 is cauterized by joule heating one of the first electrode 1151 and the second electrode 1152 by supplying power thereto. Alternatively, when the electrode is formed in only one jaw of the first jaw 1101 and the second jaw 1102, the tissue between the first jaw 1101 and the second jaw 1102 is cauterized by supplying power to and joule heating the one jaw.

At this time, a generator (not shown) configured to supply power to the electrodes may itself perform monitoring of at least some of the current, voltage, resistance, impedance, and temperature, and may stop the power supply when the cauterization is completed. Alternatively, the processor of the manipulation part 200, which controls the power supplied to the electrode, may perform cauterization based on a current value (including a momentary current value) corresponding to the power. At this time, the current value is affected by at least some of the voltage, resistance, impedance, and temperature, allowing the processor to predict whether cauterization has been completed without a separate temperature sensor or monitoring device.

In the state in which the cautery is completed as described above, when the blade wire 307 moves sequentially in the directions of an arrow A1 of FIG. 34 and an arrow A2 of FIG. 35, the blade 1175 coupled to the blade wire 307 moves from the first position at the proximal end 1105 of the end tool 1100 toward the third position at the distal end 1104 of the end tool 1100, reaching the positions in FIGS. 34 and 35 in turn.

As such, the blade 1175 cuts the tissue between the first jaw 1101 and the second jaw 1102 while moving in the X-axis direction.

However, it is to be understood that the linear motion of the blade 1175 here does not mean a motion in a completely straight line, but rather means a motion of the blade 1175 to the extent that the blade 1175 is able to cut the tissue while achieving a linear motion when viewed as a whole, even though the motion is not in a completely straight line, for example, the middle part of the straight line is bent by a certain angle or there is a section having a gentle curvature in a certain section.

Meanwhile, in this state, when the blade wire 307 is pulled in the opposite direction, the blade 1175 coupled to the blade wire 307 also returns to the first position.

According to the present disclosure, the multi-joint/multi-degree-of-freedom surgical instrument capable of pitch/yaw/actuation motions may also perform cauterizing and cutting motions.

The manipulation part 200 of the surgical instrument 10 according to an embodiment of the present disclosure includes a first handle 204 that can be gripped by a user, an actuation manipulation part 203 that controls an actuation motion of the end tool 100, a yaw manipulation part 202 that controls a yaw motion of the end tool 100, and a pitch manipulation part 201 that controls a pitch motion of the end tool 100.

The manipulation part 200 may include a pulley 210, the pulley 211, a pulley 212, a pulley 213, a pulley 214, a pulley 215, a pulley 216, a pulley 217, and a pulley 218 that are related to a rotational motion of the first jaw 101. In addition, the manipulation part 200 may include the pulley 220, a pulley 221, a pulley 222, a pulley 223, a pulley 224, a pulley 225, a pulley 226, a pulley 227, and a pulley 228 that are related to a rotational motion of the second jaw 102. In addition, the manipulation part 200 may include the pulley 213, the pulley 232, a pulley 233, and a pulley 234 that are related to a pitch motion of the second jaw 102. In addition, the manipulation part 200 may include a pulley 235, which is a relay pulley disposed at some places along the bent part 402 of the connection part 400.

Here, the pulleys facing each other are illustrated in the drawings as being formed parallel to each other, but the concept of the present disclosure is not limited thereto, and each of the pulleys may be variously formed with a position and a size suitable for the configuration of the manipulation part. Further, the manipulation part 200 of an embodiment of the present disclosure may include a rotation shaft 241, a rotation shaft 242, the rotation shaft 243, a rotation shaft 244, a rotation shaft 245, and the rotation shaft 246. Here, the rotation shaft 241 may function as a manipulation part first jaw actuation rotation shaft, and the rotation shaft 242 may function as a manipulation part second jaw actuation rotation shaft. In addition, the rotation shaft 243 may function as a manipulation part yaw main rotation shaft, and the rotation shaft 244 may function as a manipulation part yaw sub-rotation shaft. In addition, the rotation shaft 245 may function as a manipulation part pitch sub-rotation shaft, and the rotation shaft 246 may function as a manipulation part pitch main rotation shaft.

The rotation shaft 241, the rotation shaft 242, the rotation shaft 243, the rotation shaft 244, the rotation shaft 245, and the rotation shaft 246 may be sequentially disposed from a distal end 205 of the manipulation part 200 toward a proximal end 206.

Each of the rotation shafts 241, 242, 243, 244, 245, and 246 may be fitted into one or more pulleys, which will be described in detail later.

The pulley 210 functions as a manipulation part first jaw actuation pulley, the pulley 220 functions as a manipulation part second jaw actuation pulley, and these components may also be collectively referred to as a manipulation part actuation pulley.

The pulley 211 and the pulley 212 function as manipulation part first jaw yaw main pulleys, the pulley 221 and the pulley 222 function as manipulation part second jaw yaw main pulleys, and these components may also be collectively referred to as a manipulation part yaw main pulley.

The pulley 213 and the pulley 214 function as manipulation part first jaw yaw sub-pulleys, the pulley 223 and the pulley 224 function as manipulation part second jaw yaw sub-pulleys, and these components may also be collectively referred to as a manipulation part yaw sub-pulley.

The pulley 215 and the pulley 216 function as manipulation part first jaw pitch sub-pulleys, the pulley 225 and the pulley 226 function as manipulation part second jaw pitch sub-pulleys, and these components may also be collectively referred to as a manipulation part pitch sub-pulley.

The pulley 217 and the pulley 218 function as manipulation part first jaw pitch main pulleys, and the pulley 227 and the pulley 228 function as manipulation part second jaw pitch main pulleys, and these components may also be collectively referred to as a manipulation part pitch main pulley.

The pulley 231 and the pulley 232 function as manipulation part pitch main pulleys, and the pulley 233 and the pulley 234 function as manipulation part pitch sub-pulleys.

The above components are categorized from the perspective of the manipulation part for each motion (pitch/yaw/actuation) as follows.

The pitch manipulation part 201 configured to control a pitch motion of the end tool 100 may include the pulley 215, the pulley 216, the pulley 217, the pulley 218, the pulley 225, the pulley 226, the pulley 227, the pulley 228, the pulley 231, the pulley 232, the pulley 233, and the pulley 234. In addition, the pitch manipulation part 201 may include the rotation shaft 245 and the rotation shaft 246. In addition, the pitch manipulation part 201 may further include a pitch frame 208.

The yaw manipulation part 202 configured to control a yaw motion of the end tool 100 may include the pulley 211, the pulley 212, the pulley 213, the pulley 214, the pulley 221, the pulley 222, the pulley 223, and the pulley 224. In addition, the yaw manipulation part 202 may include the rotation shaft 243 and the rotation shaft 244. In addition, the yaw manipulation part 202 may further include a yaw frame 207.

The actuation manipulation part 203 configured to control an actuation motion of the end tool 100 may include the pulley 210, the pulley 220, the rotation shaft 241, and the rotation shaft 242. In addition, the actuation manipulation part 203 may further include a first actuation manipulation part 251 and a second actuation manipulation part 256.

Hereinafter, each component of the manipulation part 200 will be described in more detail.

The first handle 204 may be formed to be gripped by a user with the hand, and in particular, may be formed to be grasped by the user by wrapping the first handle 204 with his/her palm. In addition, the actuation manipulation part 203 and the yaw manipulation part 202 are formed on the first handle 204, and the pitch manipulation part 201 is formed on one side of the yaw manipulation part 202. In addition, another end portion of the pitch manipulation part 201 is connected to the bent part 402 of the connection part 400.

Here, the rotation shaft 241 and the rotation shaft 242, which are actuation rotation axes, may be formed to form a predetermined angle with the XY plane on which the connection part 400 is formed. For example, the rotation shaft 241 and the rotation shaft 242 may be formed in a direction parallel to the Z-axis, and in this state, when the pitch manipulation part 201 or the yaw manipulation part 202 is rotated, a coordinate system of the actuation manipulation part 203 may change relatively. Of course, the concept of the present disclosure is not limited thereto, and the rotation shaft 241 and the rotation shaft 242 may be formed in various directions so as to be suitable for a structure of the hand of the user gripping the actuation manipulation part 203 according to an ergonomic design.

Similarly, the pulley 220, a second actuation extension part 257, and a second actuation gear 258 are fixedly coupled to each other to be rotatable together around the rotation shaft 242. Here, the pulley 220 may be configured as a single pulley or two pulleys fixedly coupled to each other.

Here, a first actuation gear 253 and the second actuation gear 258 are formed to be engaged with each other such that, when any one gear is rotated in one direction, another gear is rotated together with the one gear in a direction opposite to the one direction.

The yaw manipulation part 202 may include the rotation shaft 243, the pulleys 211 and 212, which are manipulation part first jaw yaw main pulleys, the pulleys 221 and 222, which are manipulation part second jaw yaw main pulleys, and the yaw frame 207. In addition, the yaw manipulation part 202 may further include the pulleys 213 and 214, which are manipulation part first jaw yaw sub-pulleys formed on one side of the pulley 211 and one side of the pulley 212, respectively, and the pulleys 223 and 224 that are manipulation part second jaw yaw sub-pulleys formed on one side of the pulley 221 and one side of the pulley 222, respectively. Here, the pulleys 213 and 214 and the pulleys 223 and 224 may be coupled to the pitch frame 208 to be described later.

Here, it is illustrated in the drawings that the yaw manipulation part 202 includes the pulleys 211 and 212 and the pulleys 221 and 222, wherein the pulleys 211 and 212 and the pulleys 221 and 222 are each provided with two pulleys formed to face each other and independently rotatable, but the concept of the present disclosure is not limited thereto. That is, one or more pulleys having the same diameter or different diameters may be provided according to the configuration of the yaw manipulation part 202.

In detail, the rotation shaft 243, which is a manipulation part yaw main rotation shaft, is formed on one side of the actuation manipulation part 203 on the first handle 204. At this time, the first handle 204 is formed to be rotatable around the rotation shaft 243.

Here, the rotation shaft 243 may be formed to form a predetermined angle with the XY plane on which the connection part 400 is formed. For example, the rotation shaft 243 may be formed in a direction parallel to the Z-axis, and in this state, when the pitch manipulation part 201 is rotated, a coordinate system of the rotation shaft 243 may change relatively as described above. Of course, the concept of the present disclosure is not limited thereto, and the rotation shaft 243 may be formed in various directions so as to be suitable for a structure of the hand of the user gripping the manipulation part 200 according to an ergonomic design.

Meanwhile, the pulleys 211 and 212 and the pulleys 221 and 222 are coupled to the rotation shaft 243 so as to be rotatable around the rotation shaft 243. In addition, the wire 301 or the wire 305, which is a first jaw wire, is wound around the pulleys 211 and 212, and the wire 302 or the wire 306, which is a second jaw wire, may be wound around the pulleys 221 and 222. In this case, the pulleys 211 and 212 and the pulleys 221 and 222 may each be configured as two pulleys formed to face each other and independently rotatable. Accordingly, a wire being wound and a wire being released may be wound around respective separate pulleys so that the wires may perform motions without interference with each other.

The yaw frame 207 rigidly connects the first handle 204, the rotation shaft 241, the rotation shaft 242, and the rotation shaft 243 to each other, so that the first handle 204, the yaw manipulation part 202, and the actuation manipulation part 203 are integrally yaw-rotated around the rotation shaft 243.

The pitch manipulation part 201 may include the rotation shaft 246, the pulley 217 and the pulley 218, which are manipulation part first jaw pitch main pulleys, the pulleys 227 and 228, which are manipulation part second jaw pitch main pulleys, and the pitch frame 208. In addition, the pitch manipulation part 201 may further include the rotation shaft 245, the pulleys 215 and 216, which are manipulation part first jaw pitch sub-pulleys formed on one side of the pulley 217 and one side of the pulley 218, respectively, and the pulleys 225 and 226, which are manipulation part second jaw pitch sub-pulleys formed on one side of the pulley 227 and one side of the pulley 228, respectively. The pitch manipulation part 201 may be connected to the bent part 402 of the connection part 400 through the rotation shaft 246.

In detail, the pitch frame 208 is a base frame of the pitch manipulation part 201, and the rotation shaft 243 is rotatably coupled to one end portion thereof. That is, the yaw frame 207 is formed to be rotatable around the rotation shaft 243 with respect to the pitch frame 208.

As described above, since the yaw frame 207 connects the first handle 204, the rotation shaft 243, the rotation shaft 241, and the rotation shaft 242 to each other, and the yaw frame 207 is also axially coupled to the pitch frame 208, when the pitch frame 208 is pitch-rotated around the rotation shaft 246, the yaw frame 207 connected to the pitch frame 208, the first handle 204, the rotation shaft 241, the rotation shaft 242, and the rotation shaft 243 are pitch-rotated together with the pitch frame 208. That is, when the pitch manipulation part 201 is rotated around the rotation shaft 246, the actuation manipulation part 203 and the yaw manipulation part 202 are rotated together with the pitch manipulation part 201. In other words, when a user pitch-rotates the first handle 204 around the rotation shaft 246, the actuation manipulation part 203, the yaw manipulation part 202, and the pitch manipulation part 201 are moved together with the first handle 204.

The pulleys 217 and 218 and the pulleys 227 and 228 are coupled to the rotation shaft 246 so as to be rotatable around the rotation shaft 246 of the pitch frame 208.

Here, the pulley 217 and the pulley 218 may be formed to face each other so as to be independently rotatable. Accordingly, a wire being wound and a wire being released may be wound around respective separate pulleys so that the wires may perform motions without interference with each other. Similarly, the pulley 227 and the pulley 228 may also be formed to face each other so as to be independently rotatable. Accordingly, a wire being wound and a wire being released may be wound around respective separate pulleys so that the wires may perform motions without interference with each other.

A motion of each of the wires 303 and 304, which are pitch wires, is described as follows.

A pulley 131, which is an end tool pitch pulley, is fixedly coupled to the end tool hub 1160 in the end tool 100, and the pulley 231 and the pulley 232, which are manipulation part pitch pulleys, are fixedly coupled to the pitch frame 208 in the manipulation part 200. In addition, these pulleys are connected to each other by the wires 303 and 304, which are pitch wires, so that a pitch motion of the end tool 100 may be performed more easily according to the pitch manipulation of the manipulation part 200. Here, the wire 303 is fixedly coupled to the pitch frame 208 via the pulley 231 and the pulley 233, and the wire 304 is fixedly coupled to the pitch frame 208 via the pulley 232 and the pulley 234. That is, the pitch frame 208 and the pulleys 231 and 232 are rotated together around the rotation shaft 246 by the pitch rotation of the manipulation part 200, and as a result, the wires 303 and 304 are also moved, and thus, a driving force of additional pitch rotation may be transmitted separately from the pitch motion of the end tool 100 by the wire 301, the wire 302, the wire 305, and the wire 306, which are jaw wires.

A connection relationship of each of the first handle 204, the pitch manipulation part 201, the yaw manipulation part 202, and the actuation manipulation part 203 is summarized as follows. The rotation shafts 241 and 242, the rotation shaft 243, the rotation shaft 244, the rotation shaft 245, and the rotation shaft 246 may be formed on the first handle 204. In this case, since the rotation shafts 241 and 242 are directly formed on the first handle 204, the first handle 204 and the actuation manipulation part 203 may be directly connected to each other. In addition, since the rotation shaft 243 is directly formed on the first handle 204, the first handle 204 and the yaw manipulation part 202 may be directly connected to each other. On the other hand, since the pitch manipulation part 201 is formed on one side of the yaw manipulation part 202 so as to be connected to the yaw manipulation part 202, the pitch manipulation part 201 is not directly connected to the first handle 204, and the pitch manipulation part 201 and the first handle 204 may be formed to be indirectly connected to each other via the yaw manipulation part 202.

Continuing to refer to the drawings, in the surgical instrument 10 according to an embodiment of the present disclosure, the pitch manipulation part 201 and the end tool 100 may be formed on the same or parallel axis (X-axis). That is, the rotation shaft 246 of the pitch manipulation part 201 is formed at one end portion of the bent part 402 of the connection part 400, and the end tool 100 is formed at another end portion of the connection part 400.

In addition, one or more relay pulleys 235 configured to change or guide paths of the wires may be disposed at some places along the connection part 400, particularly in the bent part 402. As at least some of the wires are wound around the relay pulleys 235 to guide the paths of the wires, these wires may be disposed along a bent shape of the bent part 402.

Here, in the drawings, it is illustrated that the connection part 400 is formed to be curved with a predetermined curvature by having the bent part 402, but the concept of the present disclosure is not limited thereto, and the connection part 400 may be formed linearly or to be bent one or more times as necessary, and even in this case, it may be said that the pitch manipulation part 201 and the end tool 100 are formed on substantially the same axis or parallel axes. In addition, although FIG. 7 illustrates that each of the pitch manipulation part 201 and the end tool 100 is formed on an axis parallel to the X-axis, the concept of the present disclosure is not limited thereto, and the pitch manipulation part 201 and the end tool 100 may be formed on different axes.

Actuation, yaw, and pitch motions in the present embodiment will be described as follows.

First, the actuation motion will be described later.

When a user inserts his or her finger into a finger ring formed on an actuation lever 261 and rotates the actuation lever 261 using his or her finger, an actuation pulley 262, which is fixedly coupled to the actuation lever 261, rotates around the rotation shaft 241.

At this time, the wire 301 and the wire 305, each with one end portion fixedly coupled to and wound around the pulley 262, and the wire 302 and the wire 306, each with one end portion fixedly coupled to and wound around the same pulley 262, move as the pulley 262 rotates. Here, although the wires 301, 302, 305, and 306 are all coupled to one actuation pulley 262, the movement of each wire varies depending on the direction in which each wire is wound around the pulley 262 as the pulley rotates. This will be described in detail later.

In addition, a rotating force is transmitted to the end tool 1100 through the power transmission part 300, and two jaws 1103 of the end tool 1100 perform the actuation motion.

Here, as described above, the actuation motion refers to a motion in which the two jaws 1101 and 1102 are splayed or closed while being rotated in opposite directions. That is, when the actuation lever 261 of the actuation manipulation part 203 is rotated in a direction close to the first handle 204, the first jaw 1101 is rotated in the counterclockwise direction, and the second jaw 1102 is rotated in the clockwise direction, thereby closing the end tool 1100. On the contrary, when the actuation lever 261 of the actuation manipulation part 203 is rotated in a direction away from the first handle 204, the first jaw 1101 is rotated in the clockwise direction, and the second jaw 1102 is rotated in the clockwise direction, thereby opening the end tool 1100.

Next, the yaw motion will be described later.

When a user rotates the first handle 204 around the rotation shaft 243 while holding the first handle 204, the actuation manipulation part 203 and the yaw manipulation part 202 are yaw-rotated around the rotation shaft 243. That is, when the actuation pulley 262, to which the wires 301 and 305 are fixedly coupled, is rotated around the rotation shaft 243, the wires 301 and 305 wound around the pulleys 213 and 214 are moved. At this time, one of the wires 301 and 305 is wound around the pulley 213 or the pulley 214, and another one of the wires 301 and 305 is unwound from the pulley 213 or the pulley 214. Similarly, since the wire 302 and the wire 306 are also fixedly coupled to the actuation pulley 262, when the actuation pulley 262 rotates around the rotation shaft 243, the wire 302 and the wire 306 wound around the pulley 223 and the pulley 224 will move. At this time, one of the wires 302 and 306 is wound around the pulley 223 or 224, and another one of the wires 302 and 306 is wound around the pulley 223 or 224, and at this time, the wires 301 and 305 connected to the first jaw 1101 and the wires 302 and 306 connected to the second jaw 1102 are wound around the pulleys 213 and 214 and the pulleys 223 and 224 so that the first jaw 1101 and the second jaw 1102 rotate in the same direction during the yaw rotation. In addition, a rotating force is transmitted to the end tool 1100 through the power transmission part 300, and thus a yaw motion in which two jaws 1103 of the end tool 1100 are rotated in the same direction is performed.

At this time, since the yaw frame 207 connects the first handle 204, the rotation shaft 241, the rotation shaft 242, and the rotation shaft 243 to each other, the first handle 204, the yaw manipulation part 202, and the actuation manipulation part 203 are rotated together around the rotation shaft 243.

Next, the pitch motion will be described later.

When a user rotates the first handle 204 around the rotation shaft 246 while holding the first handle 204, the actuation manipulation part 203, the yaw manipulation part 202, and the pitch manipulation part 201 are pitch-rotated around the rotation shaft 246. That is, when the actuation pulley 262, to which the wires 301 and 305 are fixedly coupled, is rotated around the rotation shaft 246, the wires 301 and 305 wound around the pulleys 219 and 220 are moved. Similarly, when the actuation pulley 262, to which the wire 302 and the wire 306 are fixedly coupled, is rotated around the rotation shaft 246, the wires 302 and 306 wound around the pulleys 229 and 230 are moved. At this time, the wires 301 and 305 and the wires 302 and 306, which are jaw wires, are respectively wound around the pulleys 219 and 220 and the pulleys 229 and 230, which are manipulation part pitch main pulleys, so that the wires 301 and 305, which are first jaw wires, move in the same direction and the wires 302 and 306, which are second jaw wires, move in the same direction to pitch-rotate the first jaw 1101 and the second jaw 1102. In addition, a rotating force is transmitted to the end tool 1100 through the power transmission part 300, and two jaws 1103 of the end tool 1100 perform the pitch motion.

At this time, since the pitch frame 208 is connected to the yaw frame 207, and the yaw frame 207 connects the first handle 204, the rotation shaft 241, the rotation shaft 242, and the rotation shaft 243 to each other, when the pitch frame 208 is rotated around the rotation shaft 246, the yaw frame 207, the first handle 204, the rotation shaft 241, the rotation shaft 242, and the rotation shaft 243 connected to the pitch frame 208 are rotated together with the pitch frame 208. That is, when the pitch manipulation part 201 is rotated around the rotation shaft 246, the actuation manipulation part 203 and the yaw manipulation part 202 are rotated together with the pitch manipulation part 201.

In summary, in the surgical instrument 10 according to an embodiment of the present disclosure, the pulleys are formed on respective joint points (an actuation joint, a yaw joint, and a pitch joint), the wires (the first jaw wire or the second jaw wire) are wound around the pulleys, the rotational manipulations (actuation rotation, yaw rotation, and pitch rotation) of the manipulation part cause the movement of each wire, which in turn induces the desired motion of the end tool 1100. Furthermore, the auxiliary pulley may be formed at one side of each of the pulleys, and the wire may not be wound several times around one pulley due to the auxiliary pulley.

Hereinafter, a method of performing cauterization using the surgical instrument 10 according to an embodiment of the present disclosure will be described. For convenience of description, it is assumed that the electrode refers to the second electrode 1152 formed on the second jaw 1102, and that the electrode is not formed on the first jaw 1101, but it should be noted that both the first electrode 1151 and the second electrode 1152 can be formed on the end tool 100.

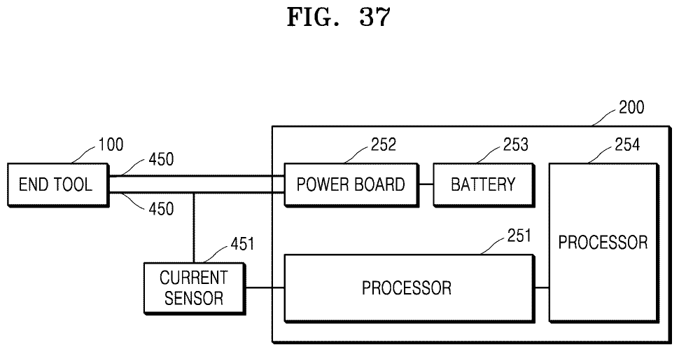

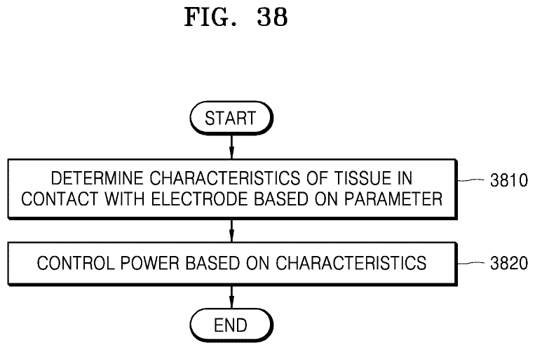

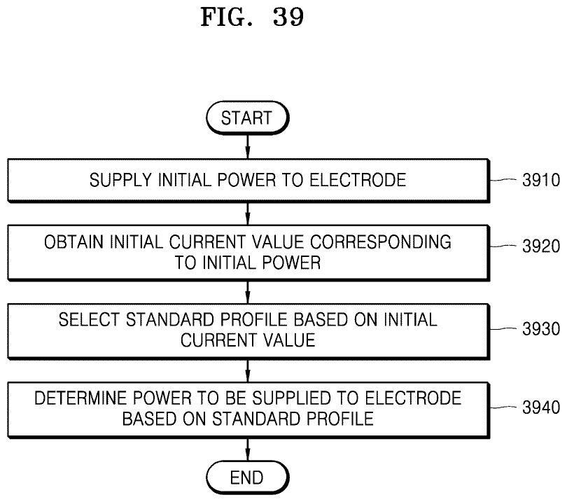

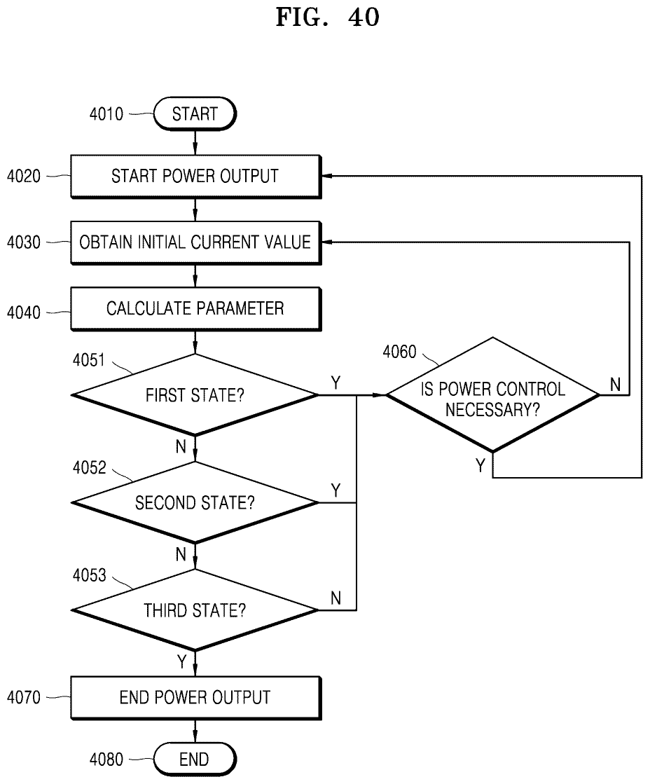

FIG. 36 is a flowchart of a method of controlling power according to an embodiment of the present disclosure. FIG. 37 is a block diagram of a surgical instrument according to an embodiment of the present disclosure. FIG. 38 is a flowchart of a method of controlling power based on moisture states according to an embodiment of the present disclosure. FIG. 39 is a flowchart of a method of determining power to be supplied to the electrode based on a standard profile according to an embodiment of the present disclosure. FIG. 40 is a flowchart of a method of controlling power according to another embodiment of the present disclosure.

Referring to FIG. 36, in operation 3610, the processor included in the manipulation part 200 (hereinafter referred to as “processor”) may obtain a momentary current value corresponding to power supplied at each of at least two or more time points among a plurality of time points included within a predetermined time period.

In an embodiment, the momentary current value corresponding to the power may refer to a measured value of a current flowing through an electric wire electrically connecting a power board to the electrode. Specifically, the processor may obtain the momentary current value corresponding to the power supplied to the electrode by obtaining a current value measured by the current sensor disposed on the electric wire electrically connecting the power board to the electrode. When there are a plurality of electrodes (for example, the first electrode 1151 and the second electrode 1152), there may be a plurality of electric wires electrically connecting the power board to the electrodes, each corresponding to a respective electrode.

Meanwhile, as described above, the power supplied to the electrode may generate joule heating in the electrode. Joule heating refers to heat generated when electrical energy is converted into thermal energy due to an electrical resistance of the electrode as a current flows through the electrode. In other words, when power is supplied to the electrode, thermal energy may be generated due to the electrical resistance of the element itself. In this case, the electrode may generate heat in an amount proportional to the square of a current value. Accordingly, the processor may predict a temperature of the electrode or the tissue in contact with the electrode using the current value corresponding to the power supplied to the electrode.

Referring to FIG. 37, the current sensor 451 is illustrated, which is disposed on an electric wire 450 that electrically connects the end tool 100 (specifically, the electrode formed in the jaw of the end tool) to a power board 252 included in the manipulation part. Although a plurality of electric wires 450 are illustrated in FIG. 37, this is only one embodiment, and the number of the electric wires 450 and the number of the current sensors 451 are not limited to the form shown in FIG. 37.

In an embodiment, the electrode may be a material that generates heat due to the element's own resistance as a current passes therethrough. As an example, the electrode may be a ceramic heating element. The ceramic heating element is an electrode used to convert supplied power, i.e., electrical energy, into heat. Ceramic materials can provide electrical safety due to their high thermal resistance and electrical insulation characteristics, in particular, ceramics themselves can generate significant heat even at relatively low power or current due to their high resistance. In addition, the ceramic heating element may have positive temperature coefficient (PTC) characteristics that allow the ceramic heating element to self-regulate the temperature thereof by increasing resistance as the temperature rises. Thus, the electrode can be made of ceramic materials to increase energy efficiency and prevent overheating.

In an embodiment, the manipulation part 200 may further include a battery 253 configured to store electrical energy and the power board 252 configured to convert the stored electrical energy to supply power to the electrode. As described above, the surgical instrument 10 may be driven using the electrical energy stored in the battery 253 without a separate connector or external power source. That is, as an example, the surgical instrument 10 may be implemented as a hand-held type device by including the battery 253. The power board 252 may receive electrical energy from the battery 253 and apply power to the end tool 100, that is, the electrode. At this time, the power applied to the electrode may be in the form of a voltage (e.g., a direct current (DC) voltage).

In an embodiment, the manipulation part 200 may include a processor 251 configured to control power. At this time, the processor 251 may be a microprocessor.

Returning to FIG. 36 again, in operation 3620, the processor may control the power to ensure that the electrode performs cauterization on the basis of a parameter calculated based on the momentary current values. In an embodiment, the processor may determine characteristics of the tissue based on the parameter and control the power based on the characteristics of the tissue. Hereinafter, this will be described with reference to FIG. 38.

Referring to FIG. 38, in operation 3810, the processor may calculate a parameter from the momentary current values. For example, the parameter may be a rate of change of the current value over time. In the following description, the rate of change of the current value over time is referred to as a current gradient, and the current gradient indicates how quickly the current value changes over time.

As an example, when the current value over time is expressed as a function I(t), the processor may calculate the current gradient as the derivative of the current value I(t) at a time t. As another example, the processor may calculate the current gradient by dividing the change in the current value over a predetermined sampling time (e.g., 10 ms) by the sampling time. As another example, the processor may graph the current values that change over time, approximate the graph to a linear function, and calculate the slope of the linear function as the current gradient. However, the method by which the device calculates the current gradient using the change in current value over time is not limited thereto. Meanwhile, the current gradient may have the unit of amperes per second (A/s).

In an embodiment, the device may determine a moisture state of the tissue at least one time point included within the predetermined time period on the basis of the parameter calculated based on the momentary current values. That is, the above-described characteristics of the tissue may include the moisture state of the tissue.

In an embodiment, the moisture state may include at least one state that occurs during the process of removing moisture contained in the tissue (e.g., blood or moisture contained in the blood) over the predetermined time period.

For example, the moisture state may include a first state. The first state is an initial stage necessary for moisture to evaporate and includes a state in which the moisture is heated and the temperature increases. Specifically, in the first state, the water molecules in the moisture may receive thermal energy, which can lead to an increase in their kinetic energy. At this time, the heat source may be the electrode.

In addition, the moisture state may include a second state. The second state includes a state in which the water molecules in the moisture, having been heated and gained sufficient kinetic energy, vaporize from a liquid state to a gas state and move into the air. Specifically, the water molecules in the moisture in the second state may receive sufficient thermal energy from the heat source to overcome the surface tension of the liquid and escape from the liquid surface.

In an embodiment, the moisture in the second state may experience a temperature drop due to latent heat of evaporation. The latent heat of evaporation is the thermal energy absorbed by the water molecules when the water molecules change from a liquid state to a gas state. For example, latent heat of evaporation for water is 2260 KJ/kg. As the latent heat of evaporation occurs in the moisture in the second state, the current gradient corresponding to the tissue in the second state may only show a consistent value or changes within a predetermined range. That is, as most of the thermal energy supplied from the electrode, which is the heat source, contributes to the latent heat of evaporation of the moisture in the second state, the temperature change of the moisture in the second state becomes negligible or small, resulting in little or no change in the current gradient, so that the current gradient may only change within a predetermined range.

In addition, the moisture state may include a third state in which the temperature of the moisture increases. The third state includes a state in which the moisture in the tissue has either fully or mostly evaporated. Accordingly, the power (or current) required to increase the temperature of the tissue in the third state is lower than that in the first state. Accordingly, a second rate of change of the temperature of the moisture in the third state may be less than a first rate of change of the temperature of the moisture in the first state. Meanwhile, when the power is continuously supplied to the tissue in the third state, the physical and/or chemical characteristics of the tissue may change. That is, by adjusting the time for supplying power to the tissue in the third state, the tissue may be heated until the desired physical and/or chemical characteristics of the tissue are achieved.

In an embodiment, the processor may determine the moisture state of the tissue in contact with the electrode based on the current gradient. Specifically, when the processor supplies power to the electrode in contact with the tissue, an initial current value is determined based on the initial impedance of the tissue. At this time, as the moisture is removed and evaporates from the tissue during the moisture removal process, the impedance of the tissue increases, but the rate of this increase tends to decrease over time.

In an embodiment, the processor may determine the moisture state of the tissue in contact with the electrode to be any one of the first to third states based on the current gradient.

As an example, the processor may determine the moisture state to be the first state based on the result of comparing the current gradient with a first threshold value. Specifically, the processor may determine the moisture state to be the first state when the current gradient is greater than or equal to the first threshold value. That is, when the tissue has a current change rate greater than the first threshold value, the processor may determine that the tissue is in the first state in which the temperature increases to cause moisture to evaporate.

As another example, the processor may determine the moisture state to be the second state based on the result of comparing the current gradient with the first threshold value and a second threshold value. Specifically, the processor may determine the moisture state to be the second state when the current gradient is less than the first threshold value and greater than or equal to the second threshold value. At this time, the second threshold value may be a value less than the first threshold value. As the temperature of the tissue increases in the first state, the impedance of the tissue also increases, and when the current gradient becomes less than the first threshold value, the processor may determine that the moisture in the tissue has begun to evaporate and determine that the moisture state is in the second state. For example, the current gradient of the tissue in the second state may exhibit only a consistent value or changes within a predetermined range due to the latent heat of evaporation. Thus, for example, the processor may determine the moisture state to be the second state when a change in current gradient is less than a certain percentage (e.g., 5%). The change in the current gradient is a rate of change of the current gradient over time, and an embodiment the same as the above-described embodiment for calculating the rate of change of current over time may be applied.

As another example, the processor may determine the moisture state to be the third state based on the result of comparing the current gradient with the second threshold value. Specifically, the processor may determine the moisture state to be the third state when the current gradient is less than the second threshold value. That is, when the moisture in the tissue has completely evaporated after passing through the second state, the impedance of the tissue may increase rapidly, and the current gradient may decrease. Accordingly, the processor may determine the moisture state to be the third state when the current gradient is less than the second threshold value.

In an embodiment, the processor may predict the temperature of the tissue based on the current gradient. For example, the processor may predict the temperature of the tissue by estimating the thickness and impedance of the tissue based on the initial current value and selecting a temperature profile of the tissue corresponding to the current gradient.

In another embodiment, the processor may integrate the obtained current values to calculate the power over time, and then integrate the power again to calculate an amount of energy. At this time, the processor may predict the specific heat of the tissue based on at least one of the predicted thickness and impedance of the tissue, which are predicted based on the initial current value. Thereafter, the processor may predict a temperature change of the tissue based on the amount of energy and the specific heat of the tissue.

That is, the processor may predict not only the moisture state of the tissue but also the temperature of the tissue based on the current gradient, and these predictions can be utilized in various industries.

In operation 3820, the processor may control the power based on the characteristics of the tissue.

In an embodiment, the processor may determine power to be supplied to the electrode based on a standard profile. Hereinafter, this will be described in detail with reference to the flowchart of FIG. 39.

Referring to FIG. 39, in an embodiment, the processor may select a standard profile for determining power to be supplied to the electrode based on initial power.

Specifically, in operation 3910, the processor may apply the initial power to the electrode. The initial power may refer to an initial value of the power supplied to the electrode.

Thereafter, in operation 3920, the processor may obtain an initial current value corresponding to the initial power. The initial current value corresponding to the initial power may refer to a current flowing through the electric wire that directly connects the power board to the electrode. Specifically, the processor may obtain the initial current value corresponding to the initial power supplied to the electrode by obtaining the current measured by the current sensor disposed on the electric wire that directly connects the power board to the electrode.

In operation 3930, the processor may select a standard profile based on the initial current value. Specifically, the processor may predict a thickness of the tissue based on the initial current value and select a standard profile corresponding to the thickness of the tissue. For example, when the tissue is thick, the tissue has a high impedance, resulting in a low current value, and in contrast, when the tissue is thin, the tissue has a low impedance, resulting in a high current value. Accordingly, the processor may calculate an impedance from the obtained initial current value and predict a thickness of the tissue based on the impedance.

For example, consider a current gradient of a first arbitrary tissue with an initial current value of s₁ at a specific time t₀, and a current gradient of a second arbitrary tissue, different from the first tissue, with an initial current value of s₂ at a specific time t₀. According to the aforementioned content, since the initial current of 51 for the first tissue is higher than the initial current of 52 for the second tissue, the processor can predict that the thickness of the first tissue is less than the thickness of the second tissue.

In an embodiment, the processor may select a standard profile that corresponds to the predicted thickness of the tissue. The standard profile may refer to a profile of the current gradient expected based on the initial current and the predicted thickness of the tissue. Thereafter, the processor may determine power to be supplied to the electrode based on the standard profile.

In operation 3940, the processor may determine power to be supplied to the electrode based on the standard profile.

In an embodiment, the processor may calculate a change in current gradient over time. Since the current gradient is an amount of change in the current value over time, the change in the current gradient may be an amount of change in the current gradient over time. Accordingly, the method used by the processor to calculate the current gradient may be applied without change to the method used to calculate the change in the current gradient.

In an embodiment, the processor may increase or decrease the power when the change in the current slope is different from the standard profile by a predetermined value or more. That is, when it is determined that a current gradient similar to the standard profile does not appear even after a predetermined time has passed, the processor may increase or decrease the power. For example, when the change in the current gradient is less rapid compared to the standard profile, it indicates that the process of removing moisture from the tissue is proceeding more slowly than expected, and thus the processor may increase the power Similarly, when the change in current gradient is rapid compared to the standard profile, it indicates that the process of removing moisture from the tissue is progressing faster than expected, and thus the processor may reduce the power.

FIG. 40 is a flowchart of a method of controlling power according to another embodiment of the present disclosure.

In an embodiment, the surgical instrument (or the manipulation part) may be activated based on an operation signal from a user. Accordingly, the processor initiates (4010) an algorithm for supplying power to the electrode.

In an embodiment, the processor outputs (4020) power to the electrode for a predetermined time period. As an example, the processor may output (4020) initial power to the electrode. Alternatively, as an example, the processor may output (4020) controlled (i.e., increased or decreased) power to the electrode.

In an embodiment, the processor may obtain (4030) a momentary current value corresponding to the power supplied to the electrode. In addition, the processor may calculate (4040) a parameter using the momentary current values. For example, the parameter could be a current gradient.

In an embodiment, the processor may determine a moisture state of the tissue in contact with the electrode based on the parameter. At this time, the processor may determine (4060) whether power control is necessary based on the moisture state. In an embodiment, the processor may control the power based on the result of the determination (4060).

As an example, the processor may determine (4051) whether the moisture state of the tissue is the first state, and determine (4060) whether power control is necessary when it is determined to be the first state, and determine (4052) whether the moisture state of the tissue is the second state when it is determined that the moisture state of the tissue is not the first state. As another example, the processor may determine (4052) whether the moisture state of the tissue is the second state, and determine (4060) whether power control is necessary when it is determined to be the second state. As another example, the processor may determine (4053) whether the moisture state of the tissue is the third state, and determine (4060) whether power control is necessary when it is determined not to be the third state. For example, the processor may determine (4060) whether power control is necessary based on the standard profile.

In an embodiment, when it is determined that power control is necessary, the processor may return to operation 4020 and output (4020) a controlled power to the electrode. On the contrary, when it is determined that power control is not necessary, the processor may return to operation 4030 to obtain a current value corresponding to the power supplied to the electrode.

As described above, the processor may repeat the above-described operations until the moisture state is determined to be the preset target state. Specifically, the processor may repeat all or some of the operations, including obtaining (4030) the momentary current values, calculating (4040) the parameter, determining (4051, 4052, and 4053) the moisture states, determining (4060) whether power control is necessary, and controlling the power or outputting (4020) the controlled power.

In an embodiment, the processor may terminate (4070) the supply of the power in response to determining that the moisture state of the tissue has reached the preset target state. As an example, when it is determined that the moisture state of the tissue is the third state, the processor may terminate (4070) the supply of the power. However, the preset target state being the third state is just one example, and the present disclosure is not limited thereto.

In an embodiment, the processor terminates (4080) the algorithm for supplying power to the electrode as the supply of the power is terminated (4070).

In an embodiment, a user may set a target state according to the desired degree of tissue deformation, and the processor may repeat the above-described processes until the target state preset by the user is determined. At this time, the tissue may be in contact with the electrode and its temperature may be raised until the state of the moisture in the tissue (e.g., blood) reaches the target state. That is, when the tissue is a blood vessel, the protein structure within the blood vessel may collapse (melt), causing the vessel walls to bond together, and as the moisture inside the vessel decreases, the vessel walls may further collapse, leading to the sealing of the blood vessel. Accordingly, the surgical instrument according to an embodiment of the present disclosure can safely seal blood vessels even without using a separate temperature sensor. Hereinafter, a sealing manipulation part controlled by the processor for sealing the tissue through cauterization and an implementation thereof will be described.

Hereinafter, a scaling manipulation part 270 of the surgical instrument 10 according to an embodiment of the present disclosure will be described.

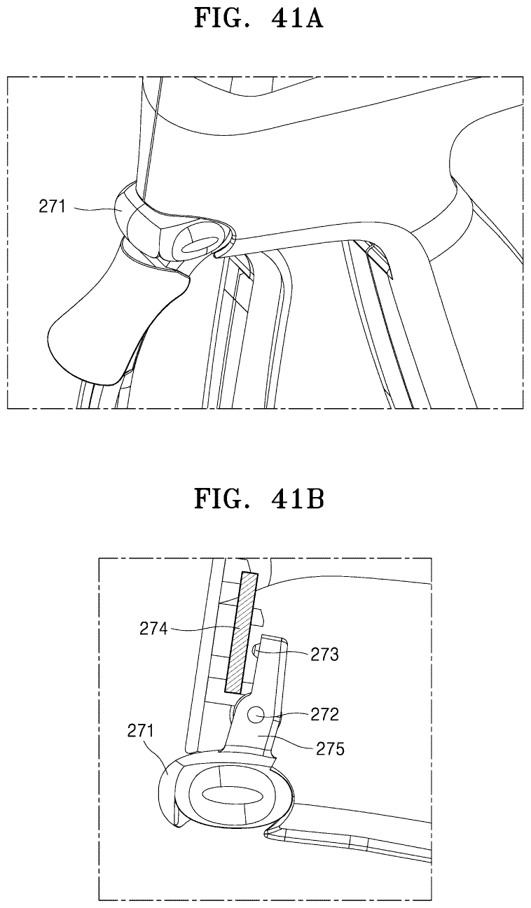

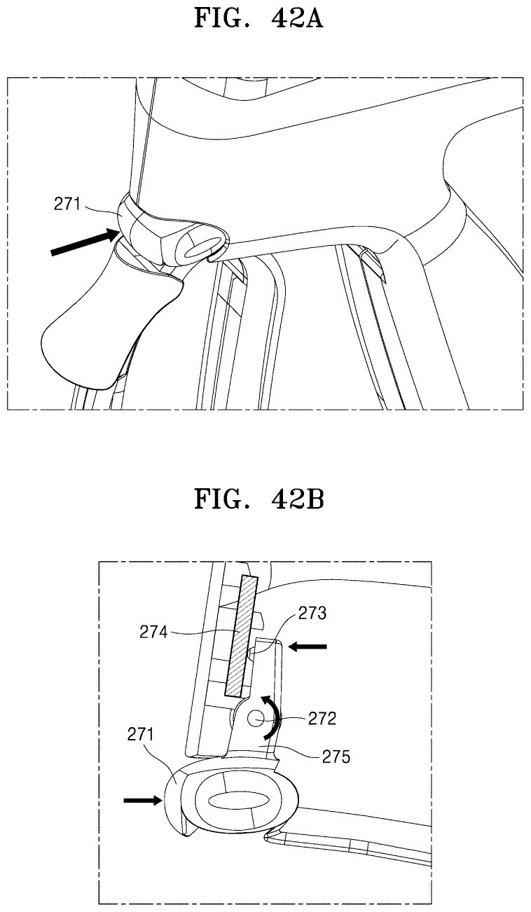

FIGS. 41 and 42 are perspective views illustrating the operation of a sealing button of the surgical instrument shown in FIG. 7. FIG. 41A is a perspective view illustrating the sealing manipulation part in a state in which the sealing button is not operated, and FIG. 41B is a partial cross-sectional view illustrating the inside of the sealing manipulation part in the state the sealing button is not operated. FIG. 42A is a perspective view illustrating the scaling manipulation part in a state in which the sealing button is being operated, and FIG. 42B is a partial cross-sectional view illustrating the inside of the sealing manipulation part in the state in which the sealing button is being operated.

Referring to FIGS. 41 and 42, the sealing manipulation part 270 may include a body part 275, a scaling button 271, a sealing rotation shaft 272, and contact parts 273 and 274.

The sealing manipulation part 270 may be formed in a region adjacent to the actuation lever 261 of the actuation manipulation part 203. In addition, the sealing manipulation part 270 may be formed on one side of a cutting lever 281 of the cutting manipulation part 280, which will be described later. Specifically, the user may operate the actuation lever 261 or the cutting lever 281 while holding the first handle 204, and the sealing button 271 may be formed in a position that facilitates its operation. For example, as shown in the drawings, the sealing button 271 may be located on the distal end side of the manipulation part 200 and may be formed so that one region of the sealing button 271 is inserted into the sealing manipulation part 270 when pressed.

In addition, the body part 275 may be formed in the shape of an elongated bar. In addition, the sealing button 271 may be formed at one end portion of the body part 275, and the contact part 273 may be formed at another end portion of the body part 275. In addition, the scaling rotation shaft 272 may be coupled to a central portion of the body part 275. Accordingly, the body part 275 is rotatable around the sealing rotation shaft 272. Meanwhile, in the sealing manipulation part 270, the contact part 274 may be formed on a surface opposite to the contact part 273.

As described above, the contact part 273 may be formed at one end portion of the body part 275 based on the sealing rotation shaft 272, and the sealing button 271 protruding outward from the manipulation part 200 may be formed at another end portion of the contact part 273.

Accordingly, when the sealing button 271 is pressed, the body part 275 may rotate around the sealing rotation shaft 272, causing the contact part 273 at the end portion of the body part 275 to move and make contact with the contact part 274.

As described above, the operation of the sealing button 271 may generate pressure or an electrical signal at the contact part 274, thereby supplying electrical energy to the first electrode 1151 and the second electrode 1152 to perform cauterization.

The present disclosure has been described above in relation to its preferred embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

According to the present disclosure, electrode temperature can be indirectly controlled by utilizing changes in current according to the amount of moisture in the tissue.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A surgical instrument comprising:
an end tool rotatable in at least one direction and including a first jaw and a second jaw opposite to the first jaw;
a manipulation part configured to control a rotational motion of the end tool; and
a connection part connecting the end tool to the manipulation part,
wherein at least one of the first jaw and the second jaw includes an electrode formed thereon and configured to receive power for a predetermined time period to be Joule heated and perform cauterization on a tissue, and
the manipulation part includes a processor configured to obtain a momentary current value corresponding to the received power at each of at least two or more time points among a plurality of time points within the predetermined time period and control the power so that the cauterization is performed based on a parameter calculated based on the momentary current values.

2. The surgical instrument of claim 1, wherein the manipulation part includes a current sensor disposed on an electric wire electrically connecting the electrode to the processor, and configured to measure the momentary current value.

3. The surgical instrument of claim 2, wherein the processor is further configured to control the power so that the electrode is heated in an open-loop manner.

4. The surgical instrument of claim 3, wherein the electrode is a ceramic heating element.

5. The surgical instrument of claim 1, wherein the end tool further includes a blade configured to, upon completion of the cauterization, cut the tissue while moving from a proximal end to a distal end of the first jaw.

6. The surgical instrument of claim 1, wherein the manipulation part includes:
a battery configured to store electrical energy; and
a power board configured to convert the stored electrical energy to supply the power to the electrode.

7. The surgical instrument of claim 6, wherein the surgical instrument is a hand-held type device.

8. The surgical instrument of claim 1, wherein the processor is further configured to determine a characteristic of the tissue in contact with the electrode based on the parameter and control the power based on the characteristic.

9. The surgical instrument of claim 8, wherein the characteristic includes a moisture state having at least one state that occurs during a process of removing moisture contained in the tissue.

10. The surgical instrument of claim 9, wherein the at least one state includes at least one of a first state in which a temperature of the moisture contained in the tissue increases, a second state in which the moisture evaporates, and a third state in which the temperature of the moisture increases, and
a first rate of change of the temperature of the moisture in the first state is greater than a second rate of change of the temperature of the moisture in the third state.

11. The surgical instrument of claim 10, wherein the processor is further configured to determine the moisture state to be the first state based on a result of comparing the parameter with a first threshold value.

12. The surgical instrument of claim 10, wherein the processor is further configured to determine the moisture state to be the second state based on a result of comparing the parameter with a first threshold value and a second threshold value,
wherein the second threshold value is less than the first threshold value.

13. The surgical instrument of claim 10, wherein the processor is further configured to determine the moisture state to be the third state based on a result of comparing the parameter with a second threshold value.

14. The surgical instrument of claim 9, wherein the processor is further configured to determine a need for control of the power based on the moisture state and control the power based on a result of the determination.

15. The surgical instrument of claim 9, wherein the processor is further configured to repeatedly calculate, determine, and control until the moisture state is determined to be a preset target state, and terminate supply of the power in response to the moisture state being determined to be the preset target state.

16. The surgical instrument of claim 8, wherein the processor is further configured to supply initial power to the electrode, obtain an initial current value corresponding to the initial power, select a standard profile based on the initial current value, and determine the power to be supplied to the electrode based on the standard profile.

17. The surgical instrument of claim 16, wherein the processor is further configured to predict a thickness of the tissue based on the initial current value and select the standard profile corresponding to the thickness of the tissue.

18. The surgical instrument of claim 16, wherein the processor is further configured to calculate an amount of change in the parameter over time, and increase or decrease the power based on the amount of change being different from the standard profile by a predetermined value or more.

19. The surgical instrument of claim 8, wherein the processor is further configured to control at least one of a voltage level and a pulse-width modulation (PWM) duty of the power.

20. The surgical instrument of claim 8, wherein the processor is further configured to predict a temperature of the tissue based on the parameter.
